(19) United States
(12) Patent Application Publication
(10) Pub. No.: US 2024/0025807 A1
(43) Pub. Date: Jan. 25, 2024

(54) PROCESSES FOR QUANTITATIVE CALCIUM SULFATE PHASE ANALYSIS AND USES THEREOF

(71) Applicant: CertainTeed Gypsum, Inc., Malvern, PA (US)

(72) Inventors: Roger Jones, Fleming Island, FL (US); Mohammad Khalkhali, Calgary (CA); Yi Lu, Edmonton (CA); Qingxia Liu, Edmonton (CA); Anirban Ghosh, Philadelphia, PA (US); Remi Lespiat, Devon, PA (US)

(21) Appl. No.: 18/225,221

(22) Filed: Jul. 24, 2023

Related U.S. Application Data
(60) Provisional application No. 63/369,168, filed on Jul. 22, 2022.

(51) Int. Cl. C04B 11/028; B01J 6/00

(52) U.S. Cl. CPC C04B 11/0281; B01J 6/004

(57) ABSTRACT

The present disclosure relates generally to processes for phase analysis of calcium sulfate materials useful in making gypsum products. In various aspects, the disclosure provides processes for providing a computer-implemented system for determining a phase content of a test calcium sulfate material; processes for determining the phase content of a test calcium sulfate material; processes for providing stucco feedstocks; and production processes for gypsum products.

### CROSS-REFERENCE TO RELATED APPLICATIONS

This application claims the benefit of priority of U.S. Provisional Patent Application No. 63/369,168, filed Jul. 22, 2022, which is hereby incorporated herein by reference in its entirety.

### BACKGROUND OF THE DISCLOSURE

### 1. Field of the Disclosure

The present disclosure relates generally to processes for phase analysis of calcium sulfate materials useful in making gypsum products.

### 2. Technical Background

Gypsum building products (e.g., wallboard, ceiling board, drywalls, or plasterboards) are panels made of a gypsum core sandwiched between two layers of paper, often referred to as facing paper, on the outside surfaces of the gypsum core. They are widely used as construction materials due to their easy fabrication, strong mechanical strength, low thermal conductivity, and soundproofing properties. The quality of gypsum boards is strongly dependent on its gypsum core, which is fabricated by the hydration of a stucco material), which primarily contains calcium sulfate hemihydrate, CaSO₄·0.5H₂O (itself known colloquially as “stucco”), into calcium sulfate dihydrate, CaSO₄·2H₂O (itself known colloquially as “gypsum”). To make better gypsum boards, higher calcium sulfate hemihydrate content in stuccos is desired to form a rigid interlocked gypsum crystal network, as well as to facilitate the control over the setting of the gypsum product.

However, real-world industrial stucco materials are always the complex mixtures of calcium sulfates with different crystalline phases, typically including calcium sulfate dihydrate, (CaSO₄·2H₂O), calcium sulfate hemihydrate, (CaSO₄·0.5H₂O), soluble calcium sulfate anhydrate (CaSO₄), inert calcium sulfate (CaSO₄), and free moisture (H₂O). The variances in stucco compositions can occur due to the use of raw gypsum materials from multiple resources, e.g., natural gypsum, flue gas desulfurization gypsum, and waste gypsum. Additionally, when producing stuccos from raw materials by calcination, the operating conditions also impact the final composition. Calcium sulfate hemihydrate contents are formed when the calcination temperature is finely controlled between 110-120° C. When the raw gypsum materials are insufficiently dehydrated, substantial amounts of calcium sulfate dihydrate remains. In contrast, when the raw gypsum materials are dried too much, fully dehydrated calcium sulfates phases (soluble calcium sulfate anhydrate, at T=120-300° C.) and inert calcium sulfate (at T>300° C.) are observed. To control the quality of gypsum products, it is desirable to have the ability to quantify each of the calcium sulfate crystalline phases in industrial stuccos and products made therefrom in a rapid, easy, and accurate manner.

Previous methodologies reported to analyze the calcium sulfate phase contents in stuccos include Ramen spectroscopy, X-ray diffraction (XRD), calorimetry, and thermogravimetric analysis (TGA). However, none of these studies can achieve a complete analysis of all calcium sulfate phases. These methods either assumed the absence of some crystalline compositions (e.g., assumed no soluble calcium sulfate anhydrate in stuccos), or characterized only certain phases in the stucco samples (e.g., only measured soluble calcium sulfate anhydrate). Unfortunately, these assumptions are not realistic for industrial stuccos which contain complex mixtures of calcium sulfate phases.

There remains a need in the art for a rapid method for a rapid and substantially complete determination of calcium sulfate phases in a stucco feedstock, for example, for a gypsum board manufacturing process.

### SUMMARY

In one aspect, the present disclosure provides a process for providing a computer-implemented system for determining a phase content of a test calcium sulfate material, the method comprising:
providing a plurality of first reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
for each of the plurality of first reference calcium sulfate samples, providing
   a first plurality of parameters associated with a dehydration weight loss curve of the first reference calcium sulfate sample, the first plurality of parameters comprising a first plurality of fit parameters associated with a fit of the dehydration weight loss curve of the first reference calcium sulfate sample, and
   a second plurality of parameters associated with a hydration temperature rise curve of the first reference calcium sulfate sample, the second plurality of parameters comprising a second plurality of fit parameters associated with a fit of the hydration curve of the first reference calcium sulfate sample,
providing the first plurality of parameters and the second plurality of parameters for each of the first reference calcium sulfate samples to a computing device;
via the computing device, correlating the first plurality of parameters and the second plurality of the parameters with the known contents of DH, HH, AIII, IN and FM of the first reference calcium sulfate samples, thereby providing a testing model that correlates the first plurality of parameters and the second plurality of parameters of a test calcium sulfate sample to contents of one or more of DH, HH, AIII, IN and FM of the test sample.

In another aspect, the disclosure provides a process as described above, further comprising
providing a plurality of second reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
for each of the plurality of second reference calcium sulfate samples, providing
   a third plurality of parameters associated with a dehydration weight loss curve of the second reference calcium sulfate sample, the third plurality of parameters comprising a third plurality of fit parameters associated with a fit of the dehydration weight loss curve of the second reference calcium sulfate sample, and
   a fourth plurality of parameters associated with a hydration temperature rise curve of the second reference calcium sulfate sample, the fourth plurality of parameters comprising a fourth plurality of fit parameters associated with a fit of the hydration curve of the second reference calcium sulfate sample;
for each of the plurality of second reference calcium sulfate samples, providing the third plurality of parameters and the fourth plurality of parameters to the testing model; and
using the testing model, determining the contents of one or more of DH, HH, AIII, IN and FM of each second reference calcium sulfate sample.

In another aspect, the disclosure provides a process for determining the phase content of a test calcium sulfate material, the method comprising:
providing a dehydration weight loss curve of the test calcium sulfate material;
providing a hydration temperature rise curve of the test calcium sulfate material;
providing
   a first plurality of parameters associated with the dehydration weight loss curve of the test calcium sulfate sample, the first plurality of parameters comprising a first plurality of fit parameters associated with a fit of the dehydration weight loss curve of the test calcium sulfate sample, and
   a second plurality of parameters associated with the hydration temperature rise curve of the test calcium sulfate sample, the second plurality of parameters comprising a second plurality of fit parameters associated with a fit of the hydration curve of the test calcium sulfate sample;
providing a testing model (e.g., developed using the methods described above) that correlates the first plurality of parameters and the second plurality of parameters of a test calcium sulfate sample to contents of one or more of DH, HH, AIII, IN and FM of the test sample;
providing the first plurality of parameters and the second plurality of parameters to the testing model; and
using the testing model, determining the contents of one or more of DH, HH, AIII, IN and FM of the test calcium sulfate sample.

In another aspect, the disclosure provides a process for providing a stucco feedstock, the process comprising
providing a first stucco material;
determining a phase content of the first stucco material according to the process described above; and
based on the determined phase content, modifying the first stucco material to provide a stucco feedstock with a desired phase content.

In another aspect, the disclosure provides a process for providing a stucco feedstock, the process comprising
calcining a first sample of a calcium sulfate material to provide a first calcined stucco using a first set of calcining parameters;
determining a phase content of the first calcined stucco according to the process described above;
based on the determined phase content, calcining a second sample of the calcium sulfate material using a second set of calcining parameters different from the first set of calcining parameters to provide the stucco feedstock.

In another aspect, the disclosure provides a process for providing a stucco feedstock, the process comprising
calcining a first calcium sulfate material to provide a first calcined stucco using a first set of calcining parameters;
determining a phase content of the first calcined stucco according to the process described above;
based on the determined phase content, selecting a second calcium sulfate material different from the first calcium sulfate material and calcining it using a second set of calcining parameters to provide the stucco feedstock.

In another aspect, the disclosure provides a production process for a gypsum product, the production process comprising:
providing a stucco feedstock by any process described above;
hydrating the first stucco feedstock in an aqueous slurry;
allowing the slurry to set; and
drying the set slurry to form the gypsum product.

In another aspect, the disclosure provides a production process for providing a gypsum product, comprising:
providing a first stucco feedstock;
determining a phase content of the first stucco feedstock by any process described above;
hydrating the first stucco feedstock in an aqueous slurry;
allowing the slurry to set; and
drying the set slurry to form the gypsum product, the process further comprising, based on the determined phase content of the first stucco feedstock, adjusting one or more parameters of the production process.

Additional aspects will be apparent to the person of ordinary skill in the art from the description herein.

### BRIEF DESCRIPTION OF THE DRAWINGS

FIG. 1 is a schematic that illustrates an example of the process for developing a testing model as described herein.

FIG. 2 is a schematic that illustrates sample preparation and data acquisition operations of various processes as described herein.

FIG. 3 is a schematic that illustrates the structure of an example of an artificial neural network for fast phase content analysis of calcium sulfate materials.

FIG. 4 is a plot of the actual phase content of the synthetic stucco samples prepared in the Example.

FIG. 5 is a plot of dehydration curves represented by the evolution of moisture changing (%) over time for representative synthetic stucco samples.

FIG. 6A is a plot of a dehydration rate as a function of time and its corresponding fitting curve using Equation 1 for a representative synthetic stucco sample. FIG. 6B is a bar graph of the R² distribution of all the fitting curves of the dehydration rates of the synthetic stucco samples prepared in the Example.

FIG. 7A is a plot of a hydration curve as a function of time and its corresponding fitting curve using Equation 2 for a representative synthetic stucco sample. FIG. 7B is a bar graph of the R² distribution of all the fitting curves for the hydration curves of the synthetic stucco samples prepared in the Example.

FIG. 8 is a representative table of the final featurized parameter matrix as described herein.

FIG. 9 is a plot of the loss values for the training dataset and the test dataset during model training of 500 epochs.

FIG. 10 are plots of the expected values of the phase content of the synthetic stucco sample and the calculated phase content based on the calculation from the machine learning model, as described herein.

FIG. 11 provides a flowchart of a CPA analysis method for phase analysis of stucco samples.

FIG. 12 provides graphs of phase analyses of single-phase samples of the particular CPA analysis example described in Example 2.

FIG. 13 provides graphs of phase analyses of the industrial stucco described in Example 2.

FIG. 14 provides compositional information for stucco mixtures made in Example 2.

FIG. 15 provides graphs comparing the expected values of DH, HH, III, and IN in the stucco mixtures of FIG. 14 as calculated from material ratios with the observed values of corresponding phases based on the phase content analysis procedure in the CPA method of Example 2.

### DETAILED DESCRIPTION

As discussed above, the present inventors have noted that quality control of stuccos is important in the production of gypsum building products. Various of the present inventors have developed a so-called “complete phase analysis” (CPA) method to quantify every calcium sulfate phase substantially present in industrial stucco based on the calculation of calcination weight loss and hydration weight gain of three pre-treated stucco samples. The CPA method features an easy experimental protocol with high accuracy, making it potentially useful for industrial quality control. However, the CPA method requires a sample preparation stage of more than 12 hours for each sample measured. Accordingly, the CPA method is very time-consuming and thus delays the data feedback to the production line. Therefore, there is a need to provide a more rapid quantitative test.

To address this need in the art, the present inventors have developed a method for rapid and quantitative analysis of calcium sulfate materials by using testing model that correlates data from two fast and simple physical analyses—weight loss upon dehydration and temperature rise upon hydration—with the content of the calcium sulfate phases substantially present in industrial stuccos: calcium sulfate dihydrate (DH), calcium sulfate hemihydrate (HH), soluble calcium sulfate anhydrite (AIII); inert calcium sulfate (IN) and free moisture (FM).

Machine learning techniques can be used to develop the testing model from a library of test data of known reference calcium sulfate samples. In the past decade, machine learning has been adopted in many areas to study the wealth of existing data in many fields, including materials science. Machine learning is a powerful tool in finding mathematical relationships between input and output datasets, especially useful when the data is too large in size or too complicated in structure for human analysis, and when the conceptualization of the mathematical relationships is not necessary. Moreover, there is no need to assume physical or chemical rules in machine learning algorithms. Instead, the relationships are derived from the reference data.

Especially, algorithms based on artificial neural networks (ANN) provide one of the most comprehensive and versatile machine learning algorithms that are generally workable for solving practical issues.

As the person of ordinary skill in the art will appreciate, ANN-based techniques can be prone to overfitting, which can make the algorithm less accurate in analyzing an unseen dataset. To avoid overfitting and improve the general applicability of the ANN techniques, common regularization protocols, such as dropout and early stopping techniques, can be applied. In a dropout technique, randomly selected neurons are ignored during training to prevent codependent neurons. In the early stopping technique, the model training is terminated when the algorithm starts to perform worse on unseen dataset. Both techniques can be effective on preventing overfitting in ANN, and both techniques are applied in this example.

Here, the measurements performed on the test calcium sulfate sample are fast to perform, generally no more than an hour, and require conventional equipment for sample treatment. The resulting data are provided to a pre-trained machine learning model to determine the calcium sulfate phases present in the test calcium sulfate sample with a high degree of accuracy. The present inventors have found that this method can be particularly useful in production facilities by providing calcium sulfate phase analysis to gypsum product production lines. Furthermore, the present inventors have found that because the method provides rapid quantitative phase analysis of gypsum products on the production line, the results of the analysis can be used in near real-time to effect changes in production, for example, in the process of providing a stucco feedstock to a gypsum product production line, or in the processing of that stucco feedstock to provide a gypsum product. For example, the particular method described with reference to the Example below, based on a few hundred reference calcium sulfate samples, determines the calcium sulfate phase content with a root-mean-square error (RMSE) of 2.2% and a free moisture calculation accuracy of 87.7% —and this accuracy may be increased over time with more training data.

Thus, the methods of the disclosure can provide fast response times to production lines to ensure the quality of the gypsum product, saving both time and cost, as well as reducing production waste.

As described above, one aspect of the present disclosure provides a process for providing a computer-implemented system for determining a phase content of a test calcium sulfate material. The process includes:
providing a plurality of first reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
for each of the plurality of first reference calcium sulfate samples, providing
   a first plurality of parameters associated with a dehydration weight loss curve of the first reference calcium sulfate sample, the first plurality of parameters comprising a first plurality of fit parameters associated with a fit of the dehydration weight loss curve of the first reference calcium sulfate sample, and
   a second plurality of parameters associated with a hydration temperature rise curve of the first reference calcium sulfate sample, the second plurality of parameters comprising a second plurality of fit parameters associated with a fit of the hydration curve of the first reference calcium sulfate sample,
providing the first plurality of parameters and the second plurality of parameters for each of the first reference calcium sulfate samples to a computing device;
via the computing device, correlating the first plurality of parameters and the second plurality of the parameters with the known contents of DH, HH, AIII, IN and FM of the first reference calcium sulfate samples, thereby providing a testing model that correlates the first plurality of parameters and the second plurality of parameters of a test calcium sulfate sample to contents of one or more of DH, HH, AIII, IN and FM of the test sample.

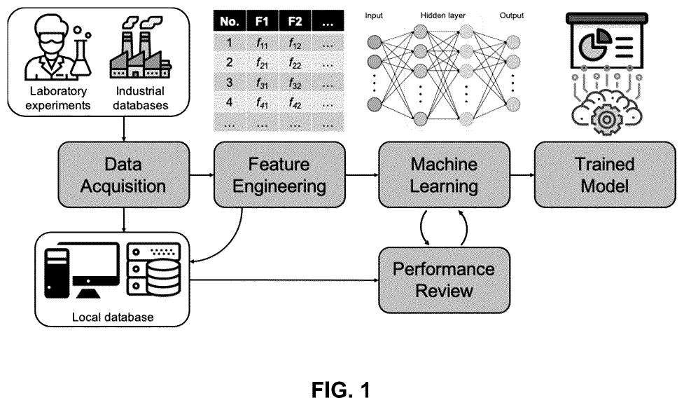

The process is based on correlation of hydration and dehydration data to phase content of a plurality of first reference calcium sulfate samples, to provide the testing model. An example of a process flow for the provision of a testing model is shown in FIG. 1. In general, the process flow includes three main primary steps: (1) data acquisition with respect to first reference calcium sulfate samples; (2) so-called feature engineering, i.e., converting that data to a set of parameters for use in correlation to phase contents of the first reference calcium sulfate samples; and (3) correlation of the parameters with the known phase content of the first reference calcium sulfate samples, for example, using machine learning techniques. In the data acquisition step, laboratory-scale or industrial-scale experiments are conducted to collect a body of data to be used for model training. In the feature engineering step, the raw experimental data collected in the first step for each first reference calcium sulfate sample are translated into well-designed featurized parameters that are easily readable in programming language. For example, the featurized parameters can include parameters developed by fitting a dehydration weight loss curve and fitting a hydration temperature rise curve, as described below. This reduces the dimensions of the input data matrix to the correlation, and thus enhances the calculation efficiency of the correlation. In the third step, the parameters are correlated with the known phase content of the first reference calcium sulfate samples. For example, machine learning can be used, for example, with training by the experimental data with certain learning rules, where an artificial neural network (ANN) algorithm is utilized to determine the relationship between the input values and the calcium sulfate phase content. Finally, (4) a validation can be used to determine the accuracy of the machine learning model and tune the training parameters in the machine learning model based on its evaluation over many iterations (or “epochs”).

Good correlations can be provided by using a relatively large number of first calcium reference samples. For example, machine learning algorithms analysis of large data sets, and machine learning models can be more accurate if a larger database is available for training purposes. For example, in various embodiments, the plurality of first calcium samples comprises at least 100 first reference calcium sulfate samples, e.g., at least 200 first reference calcium sulfate samples. In various embodiments, the plurality of first reference calcium sulfate samples comprises at least 300 first reference calcium sulfate samples, e.g., at least 400 first reference calcium sulfate samples.

First reference calcium sulfate samples having a known content of calcium sulfate dihydrate (DH), calcium sulfate hemihydrate (HH), soluble calcium sulfate anhydrite (AIII); inert calcium sulfate (IN) and free moisture (FM) can be provided, for example, by combining one or more of an industrial stucco product, phase-pure DH, phase-pure HH, phase-pure AIII and phase-pure IN. “Industrial stucco product” means a stucco product that is primarily stucco, but includes substantial amounts of one or more of DH, HH, AIII and IN. “Phase-pure” refers to samples that are at least 95 wt % of the stated calcium sulfate phase. The reference calcium sulfate samples are desirably at least 95 wt % (e.g., at least 98 wt %, or at least 98 wt %) made up of one or more of DH, HH, AIII, IN and FM. The person of ordinary skill in the art can use conventional methods to determine the phase content of the first reference calcium sulfate samples. The CPA method provided as can alternatively be used. Samples made by addition of phase-pure materials can be simply calculated by the weights of the combination of materials.

First reference calcium sulfate samples desirably substantially span the likely phase content space of the test samples to be tested. For example, in various embodiments, for at least 80% (e.g., at least 90% or at least 95%) of the plurality of first reference calcium sulfate samples, the known amount of DH is in the range of 3-20 wt %. In various embodiments, for at least 80% (e.g., at least 90% or at least 95%) of the plurality of first reference calcium sulfate samples, the known amount of HH is in the range of 50-90 wt %. In various embodiments, for at least 80% (e.g., at least 90% or at least 95%) of the plurality of first reference calcium sulfate samples, the known amount of AIII is up to 30 wt %. In various embodiments, for at least 80% (e.g., at least 90% or at least 95%) of the plurality of first reference calcium sulfate samples, the known amount of IN is in the range of 5-10 wt %.

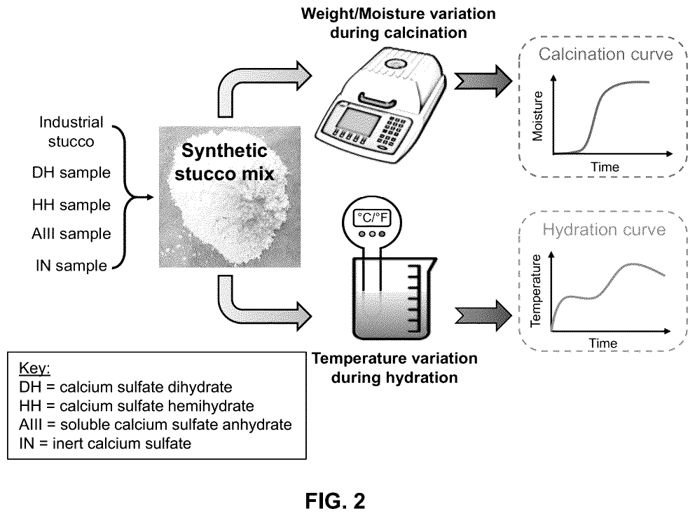

As described above, characterization data are collected for each of the plurality of first calcium reference samples. An example of a process of data acquisition is illustrated in FIG. 2. First reference calcium sulfate samples, e.g., as synthetic stucco mixtures, are analyzed for weight loss during calcination and temperature rise during hydration, providing curves for each. Substantially the same experimental technique should be used for all first reference calcium sulfate samples, to ensure that changes in experimental results are directly related to the variations of the phase content in corresponding synthetic stucco samples.

For each of the plurality of first reference calcium samples, dehydration testing is conducted to provide a dehydration weight loss curve. During dehydration, calcium sulfate dihydrate and calcium sulfate hemihydrate contents lose their water of hydration and become soluble calcium sulfate anhydrate, as shown in the following reactions:

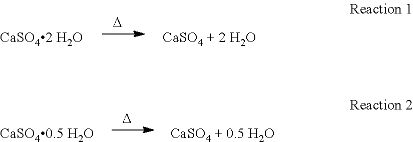

Accordingly, the weight loss during dehydration tests is directly correlated with the compositions of calcium sulfate dihydrate and calcium sulfate hemihydrate contents in the first reference calcium sulfate samples.

Dehydration testing can be conducted in a variety of manners, for example, by monitoring the weight loss of the first reference calcium sulfate samples during high-temperature dehydration using thermogravimetric analysis (TGA). For example, in various embodiments, the dehydration weight loss curve of each first reference calcium sulfate sample is provided by thermogravimetrically measuring weight loss of the first reference calcium sulfate sample at a temperature of at least 100° C., e.g., at least 200° C., or at least 250° C., e.g., in the range of 100-500° C., or 200-500° C., or 250-500° C., or 100-400° C., or 200-400° C., or 250-400° C. The dehydration weight loss can be measured, for example, over a time in the range of 5 to 30 minutes. The dehydration weight loss is desirably measured until the moisture change reaches a plateau. As used herein, a “dehydration weight loss curve” is a dataset providing weight losses as a function of time; it need not be plotted graphically. Data points can be provided at an average spacing up to 15 seconds, e.g., up to 10 seconds, or up to 5 seconds. The person of ordinary skill in the art will select a density of data that provides desired correlation results but maintains a reasonable dataset size.

Hydration testing can be conducted in a variety of manners, generally measuring the temperature rise upon hydration of the first reference calcium sulfate sample. The temperature increase is caused by the reaction of calcium sulfate hemihydrate and soluble calcium sulfate anhydrate with water and release heat, as shown in the following reactions:

CaSO₄+0.5H₂O→CaSO₄·0.5H₂O+Q Reaction 3

CaSO₄·0.5H₂O+1.5H₂O→CaSO₄·2H₂O+Q Reaction 4

where Q is the amount of heat evolved during hydration. Since both Error! Reference source not found. and Error! Reference source not found. are highly exothermic reactions, their occurrence heats the surrounding water. As with the dehydration testing, different phase contents of the first reference calcium sulfate samples result in different hydration curves, i.e., different functions of mixture temperature with time.

For example, the hydration temperature rise curve of each first reference calcium sulfate sample is provided by measuring the temperature rise as a function of time of a mixture of the first reference calcium sulfate sample and water. In various embodiments, the temperature rise is measured over a period in the range of 15 minutes to 60 minutes, e.g., 15-45 minutes. The weight ratio of first reference calcium sulfate sample to water can be, for example, in the range of 1:0.5-1:5, e.g., 1:0.5-1:2. The hydration temperature rise is desirably measured until the temperature change reaches a plateau. As used herein, a “hydration temperature rise curve” is a dataset providing weight losses as a function of time; it need not be plotted graphically. Data points can be provided at an average spacing up to 15 seconds, e.g., up to 10 seconds, or up to 5 seconds. The person of ordinary skill in the art will select a density of data that provides desired correlation results but maintains a reasonable dataset size.

The dehydration data and the hydration data will often contain many individual data points. As would be understood by the person of ordinary skill in the art, machine learning training using such a large dataset would be resource-intensive and slow. Accordingly, a feature engineering step is desirable to “translate” the raw experimental data into numerical parameters that contains the most important characteristics of the raw data concisely, but also are easily handled in the programming language. In other words, the feature engineering step can be specifically designed to reduce the computation power and increase calculation efficiency.

Accordingly, a first plurality of parameters associated with the dehydration weight loss curve of each first reference calcium sulfate sample is provided. The first plurality of parameters can include, for example, at least four parameters (e.g., 4 parameters, or 5 parameters, or 6 parameters).

The present inventors have determined that useful parameters can be provided by fitting the dehydration weight loss curve to a mathematical function. Accordingly, the first plurality of parameters include a first plurality of fit parameters associated with a fit of the dehydration weight loss curve of the first reference calcium sulfate sample. This can be, for example, a fit of the dehydration weight loss curve itself. As an alternative, the fit of the dehydration weight loss curve can be provided as a fit of the moisture change rate as a function of time, the moisture change rate being the first derivative of the dehydration weight loss curve. In various embodiments, this can be fit to a Gaussian function, for example, the equation: dehydration rate

$$( \% / s ) = a \cdot exp [ - {( \frac{{t}_{1} - b}{c} )}^{2} ] .$$

In various embodiments, the first plurality of fit parameters includes at least three fit parameters, e.g., three fit parameters, or four fit parameters, or five fit parameters.

The first plurality of parameters can also include other parameters. For example, in various embodiments, the first plurality of parameters includes a parameter associated with a maximum weight change of the dehydration weight loss curve. This can simply be the value of the maximum weight change of the dehydration weight loss curve.

Similarly, a second plurality of parameters associated with the hydration temperature rise curve of each first reference calcium sulfate sample is provided. The second plurality of parameters can include, for example, at least four parameters, e.g., at least five parameters, or at least six parameters, for example, 4 parameters, 5 parameters, 6 parameters, 7 parameters or 8 parameters.

The present inventors have determined that useful parameters can be provided by fitting the hydration temperature rise curve to a mathematical function. Accordingly, the second plurality of parameters include a second plurality of fit parameters associated with a fit of the hydration temperature rise curve of the first reference calcium sulfate sample. This can be, for example, a fit of the hydration temperature rise curve itself. In various embodiments, this can be fit to a combination of Gaussian functions, e.g., a combination of a positive-skewed Gaussian function and a non-skewed Gaussian function. As an example, the fit can to be an equation: temperature

$$change = u \cdot exp [ - {( \frac{ln ( {t}_{2} ) - v}{w} )}^{2} ] + x \cdot exp [ - {( \frac{{t}_{2} - y}{z} )}^{2} ] ,$$

e.g., where the temperature change is provided as a normalized temperature change by dividing temperature change by initial temperature. In other embodiments, the normalized temperature change can be calculated by subtracting all temperature values from the temperature at time 0. In various embodiments, the second plurality of fit parameters includes at least four fit parameters, for example, at least five fit parameters or at least six fit parameters, e.g., four fit parameters, or five fit parameters, or six fit parameters, or seven fit parameters, or eight fit parameters.

The first plurality of parameters and the second plurality of parameters are provided to a computing device, e.g., for using machine learning to develop the testing model. The person of ordinary will appreciate that the computing device can include a processor programmed to perform a machine learning algorithm. This can take the form of an appropriately-programmed general purpose computer. As would be understood by the person of ordinary skill in the art, a computing device includes any known device suitable for computing. For example, the computing device may be a user device (e.g., a device actively operated by a user), such as a mobile device (e.g., tablet computer or smartphones) or a stationary device (e.g., desktop computers or laptop computers). Such devices may include one or more processors (e.g., a central processing unit (CPU), a graphics processing unit (GPU), a network processor, and/or a form of integrated circuit or controller that performs processor operations) one or more sensors (e.g., gyroscopes, accelerometers, cameras, touchscreens, tactile buttons, keyboards, etc.), one or more network communication models (e.g., IEEE 802.11 (Wi-Fi), BLUETOOTH®, global positioning system (GPS), a wide-area wireless interface, Ethernet, Synchronous Optical Networking, digital subscriber line, etc.), and one or more digital storage systems (e.g., random access memory (RAM), read-only memory (ROM), flash memory, hard disk drives, solid state drives, compact discs (CDs), digital video disks (DVDs), and/or tape storage), all of which may be connected by a system bus or a similar mechanism. In some examples, the computing device may include other components and/or peripheral devices (e.g., keyboards, mouse, sensors, detachable storage, printers, etc.).

The machine learning algorithm can be, for example, artificial neural network algorithm, as described herein. The present inventors have determined that artificial neural network algorithms can be especially useful to map the relationship between the featurized parameters and the phase content of the first reference calcium sulfate samples. In machine learning applications, artificial neural networks decompose a complex nonlinear function into a composition of linear transformations with learnable parameters connected by nonlinear activation functions. With increasing numbers of such nonlinear transformations organized in “layers”, it is possible to efficiently learn highly complex feature spaces.

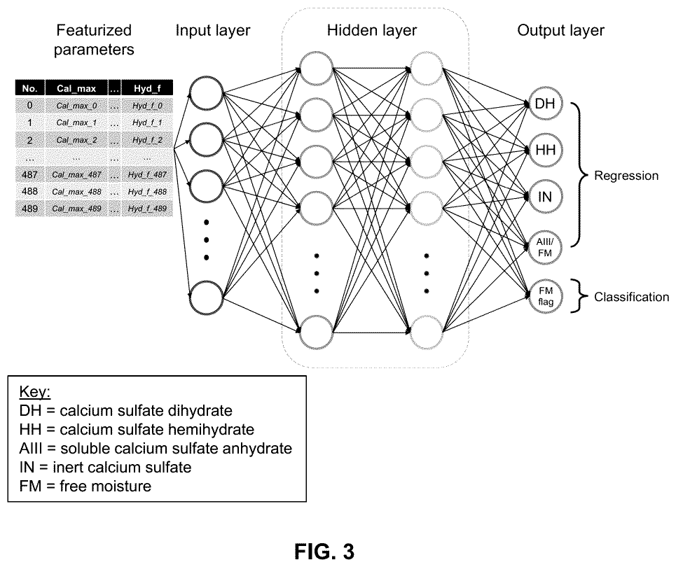

In various embodiments, the artificial neural network has an input layer comprising at least one node for each parameter of the first plurality of parameters and the second plurality of parameters; at least two hidden layers having nodes associated with the input layer; and an output layer having at least five output nodes associated with the hidden layers. One such embodiment is shown in FIG. 3, in which a large number of neurons are arranged in multiple layers in the following sequence:
(1) An input layer where the featurized parameters are fed into the artificial neural network.
(2) One or more hidden layers where neurons are interconnected.
(3) An output layer to provide calculated values.

Neurons at the input layer first receive the values of featurized parameters and then process the signals to the next connected neurons, where a weight is applied to adjust each learning process. The artificial neural network used here contained two hidden layers, where each layer has 64 neutrons. Ultimately, the featurized parameters travel from the input layer, progress through the hidden layer, and eventually reach the output layer with calculated values.

The artificial neural network algorithm is a powerful machine learning model that can be used for both regression and classification problems. Regression is a category of machine learning that estimates the relationship between some input variables and one or more numeric outputs. Classification maps the relationship between input variables with output non-numerical labels. In various embodiments, both functions can be implemented. For example, in various embodiments, the output nodes include regression nodes for each of DH, HH and IN. In various embodiments, the output nodes include two nodes that together relate to AIII and FM, e.g., a regression node for the total of AIII and FM, and a classification node classifying whether AIII or FM is present. An example is shown in FIG. 3. There, the first four nodes were designed to provide the calculated values of calcium sulfate dihydrate (DH), calcium sulfate hemihydrate (HH), inert calcium sulfate (IN), and the total amount of soluble calcium sulfate anhydrate/free moisture (AIII/FM) contents in a calcium sulfate sample, all provided by regression analysis. The classification function was introduced in the fifth node of the output layer, called the “free moisture flag”. It is known that soluble calcium sulfate anhydrate and free moisture cannot coexist in real-world samples, and, so for quantification purposes they shared the same node. The free moisture flag was used to indicate whether the value shown in the soluble calcium sulfate anhydrate/free moisture node represents an amount of soluble calcium sulfate anhydrate (when free moisture flag=0) or an amount of free moisture content (when free moisture flag=1).

Thus, using an artificial neural network or otherwise, the first plurality of parameters and the second plurality of the parameters are correlated with the known contents of DH, HH, AIII, IN and FM of the first reference calcium sulfate samples via the computing device. This provides a testing model that correlates the first plurality of parameters and the second plurality of parameters of a test calcium sulfate sample to contents of one or more of DH, HH, AIII, IN and FM of the test sample.

An artificial neural network can be trained by comparing the difference between the calculated values from the output layer and the actual values from sample measurement. The differences between calculated and reality are described by the loss value (L) as defined by the following equation: L=Σ₁^{m}(yᵢ−ŷᵢ)², in which yᵢ is the calculated value from the output layer in the i^{th} group, ŷᵢ is the real value for the i^{th} group, and m is the group of samples being fed in. Based on this definition, an artificial neural network model with a smaller value of L means a more accurate calculation with respect to the actual value. Therefore, artificial neural network training targets reducing the L value throughout a number of iterations (known as epochs), in which weight associations associated with the parameters are adjusted according to a designed learning rule. Ideally, the L value would become smaller over iterations, which means that the model calculation becomes closer to the actual values.

For example, in various embodiments, providing the testing model comprises training the artificial neural network algorithm through a plurality of epochs, e.g., at least 100 epochs, or at least 200 epochs, or at least 250 epochs. In each epoch, training the artificial neural network algorithm can include comparing differences between calculated values of the output layer and actual known values of one or more of DH, HH, IN, AIII and FM of the first reference calcium sulfate samples. For example, in response to differences between calculated values of the output layer and actual known values of one or more of DH, HH, IN, AIII and FM of the first reference calcium sulfate samples, weight associations can be adjusted with respect to one or more of the parameters of the first plurality of parameters and the second plurality of parameters.

The process can further include providing a plurality of second reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM). There can be, e.g., at least 50, at least 100, or at least 200 second reference calcium sulfate samples; the person of ordinary skill in the art will select a desired number, with reference to the description herein, that provides meaningful validation of the testing model. These second reference calcium sulfate samples can be treated in the same manner as the plurality of first reference calcium sulfate samples as described above, to provide a third plurality of parameters associated with a dehydration weight loss curve of the second reference calcium sulfate sample, the third plurality of parameters comprising a third plurality of fit parameters associated with a fit of the dehydration weight loss curve of the second reference calcium sulfate sample, and a fourth plurality of parameters associated with a hydration temperature rise curve of the second reference calcium sulfate sample, the fourth plurality of parameters comprising a fourth plurality of fit parameters associated with a fit of the hydration curve of the second reference calcium sulfate sample. Data associated with the second plurality of reference samples can be used in one or more of the epochs to test the validity of the model as existing in that epoch with respect to data that is not part of the training data set. This can guard against the complication of “overfitting,” in which the artificial neural network tends to fit closely to the training data set but fail to reliably fit with additional data. Accordingly, for each of the plurality of second reference calcium sulfate samples, the third plurality of parameters and the fourth plurality of parameters can be provided to the testing model; and using the testing model, the contents of one or more of DH, HH, AIII, IN and FM of each second reference calcium sulfate sample can be determined. This can be, for example, with respect to contents of two or more, three or more, four or more, or all five of DH, HH, AIII, IN and FM. This can be performed in a plurality of epochs, and can further include providing a loss value (L) for each epoch, and selecting as a final testing model the testing model of an epoch where the loss value is substantially minimized (e.g., within 10% of a minimum value, or within 5% of a minimum value). Thus, the person of ordinary skill in the art, by examining performance with respect to the plurality of second reference calcium sulfate samples, can select a final training model that is properly trained but not overtrained, by having low Loss Values not only for the plurality of first reference calcium sulfate samples using which the model was trained, but also for a different plurality of second reference calcium sulfate samples.

With a trained testing model in hand, e.g., prepared as described above, the phase content of unknown calcium sulfate samples can be determined quickly and easily. All that is necessary is to provide a dehydration weight loss curve and a hydration temperature rise curve for the test calcium sulfate material, parameterize the data, and apply the data to the trained model. Accordingly, another aspect of the disclosure provides a process for determining the phase content of a test calcium sulfate material, the method including:
providing a dehydration weight loss curve of the test calcium sulfate material;
providing a hydration temperature rise curve of the test calcium sulfate material;
providing
   a first plurality of parameters associated with the dehydration weight loss curve of the test calcium sulfate sample, the first plurality of parameters comprising a first plurality of fit parameters associated with a fit of the dehydration weight loss curve of the test calcium sulfate sample, and
   a second plurality of parameters associated with the hydration temperature rise curve of the test calcium sulfate sample, the second plurality of parameters comprising a second plurality of fit parameters associated with a fit of the hydration curve of the test calcium sulfate sample;
providing a testing model that correlates the first plurality of parameters and the second plurality of parameters of a test calcium sulfate sample to contents of one or more of DH, HH, AIII, IN and FM of the test sample;
providing the first plurality of parameters and the second plurality of parameters to the testing model; and
using the testing model, determining the contents of one or more of DH, HH, AIII, IN and FM of the test calcium sulfate sample.

The testing model can be provided, for example, in any manner as described above.

The test calcium sulfate sample desirably has a phase content that is within the phase content space of the first reference calcium sulfate sample serving as the basis of the training of the testing model. For example, in various embodiments, the amount of DH in the test calcium sulfate sample is in the range of 3-20 wt %. In various embodiments, the amount of HH in the test calcium sulfate sample is in the range of 50-90 wt %. in various embodiments, the amount of AIII in the test calcium sulfate sample is up to 30 wt %. In various embodiments, the amount of IN in the test calcium sulfate sample is in the range of 5-10 wt %.

The provision of the dehydration and hydration test data can be performed as described above. The person of ordinary skill in the art will appreciate that the dehydration and hydration test data should be collected in a manner similar to the data serving as the basis for the testing model. While some experimental details may differ, the method used to collect the data for the test calcium sulfate sample should yield substantially the same results. Of course, the person of ordinary skill in the art can collect data in a substantially different manner, as long as the data or the resulting parameters can be reliably transformed to a values that are substantially similar to that which would be provided using the procedures used to develop the data underlying the testing model.

Similarly, the first and second pluralities of parameters of the test calcium sulfate sample can be provided in substantially the same manner as the first and second pluralities of parameters for the first reference calcium sulfate samples used in training the testing model. The person of ordinary skill in the art will appreciate that computational details may differ, but the underlying fitting equations are desirably the same, or, at least, the resulting parameters should relate to the data in substantially the same manner.

Once determined, the determined contents of one of more of DH, HH, AIII, IN and FM of the test calcium sulfate sample can be provided to a user in any fashion. They can be displayed on a screen, sent via email or other messaging surface, and/or saved in a database.

Moreover, and most importantly, the person of ordinary skill in the art, based on the present disclosure, can use the determined contents of one or more of DH, HH, AIII, IN and FM of the test calcium sulfate sample in a variety of manners to determine and adjust various process parameters in the production of stucco feedstocks and gypsum products.

For example, the person of ordinary skill in the art, based on the present disclosure, can use measurements as described herein in the provision of stucco feedstocks, for example, suitable in the production of a gypsum product such as a gypsum building board.

For example, the information can be used as a basis for modifying a stucco material to provide a desired phase content. Accordingly, another aspect of the present disclosure is a process for providing a stucco feedstock. The process includes providing a first stucco material; determining a phase content of the first stucco material according to a process as described herein; and, based on the determined phase content, modifying the first stucco material to provide a stucco feedstock with a desired phase content. The modification can, in some embodiments, be a combination of the first stucco material with one or more second stucco materials each having a different phase content from the first stucco material, to provide a stucco feedstock with a desired phase content. A stucco production facility can have different stucco materials of different phase contents, and can mix them based on the measurements made as described herein to provide a variety of stucco feedstocks having different desired phase contents. Various second stucco materials can be, e.g., derived from material recycled from earlier processes, and/or derived from a different mineral source. In various embodiments, the modification of the first stucco material includes calcining the first stucco material; in a case where the phase content measurement suggests incomplete calcination, the first stucco material can be calcined further to provide the desired phase content. In various embodiments the modification of the first stucco material includes adding moisture to the first stucco material, e.g., in cases where the first stucco material was over-calcined to provide an undesirably high level of dehydration. In various embodiments, the modification of the first stucco material includes adjusting a particle size distribution (e.g., by adjusting grinding conditions) of the first stucco material.

As another example, the information provided by the measurement processes described herein can be used as a basis for adjusting calcination parameters for a given calcium sulfate material. For example, another aspect of the disclosure provides a process for providing a stucco feedstock includes calcining a first sample of a calcium sulfate material to provide a first calcined stucco using a first set of calcining parameters; determining a phase content of the first calcined stucco according to a process as described herein; and based on the determined phase content, calcining a second sample of the calcium sulfate material using a second set of calcining parameters different from the first set of calcining parameters to provide the stucco feedstock. Here, the measured phase content after calcination under the first set of calcination parameters can be used by the person of ordinary skill in the art to determine how to adjust the calcination parameters for a calcination of a second sample of the same material. In various embodiments, the second set of calcination parameters has a different calcination temperature program than the first set. In various embodiments, the second set of calcination parameters has a different calcination time than the first set.

A variety of calcining parameters can be adjusted, based on the determined phase content. For example, the calcining temperature can be raised, with higher temperatures providing higher rates of calcination, or lowered, with lower temperatures providing lower rates of calcination. The humidity in the calciner can be raised, with higher humidities providing lower rates of calcination, or lowered, with lower humidities providing lower rates of calcination. The throughput of the calciner can be raised, with higher throughput (i.e., less time in calciner) providing a lower degree of calcination, or lowered, with lower throughput (i.e., more time in calciner) providing a higher degree of calcination. Similarly, the calcination time can be lengthened, with a longer time providing a higher degree of calcination, or shortened, with a shorter time providing a lower degree of calcination. Calcining of the second sample to provide a higher degree of calcination than in the calcination of the first sample can desirably, e.g., provide relatively less calcium sulfate dihydrate and/or less free moisture in the stucco feedstock than in the first calcined stucco. Calcining of the second sample to provide a lower degree of calcination than in the calcination of the first sample can desirably, e.g., provide relatively less calcium sulfate anhydrate and/or relatively less inert material in the stucco feedstock than in the first calcined stucco.

As another example, the information provided by the measurement processes described herein can be used as a basis for determining an appropriate calcium sulfate feedstock for a calcination process to provide a desired stucco feedstock. For example, another aspect of the disclosure provides a process for providing a stucco feedstock, the process including: calcining a first calcium sulfate material to provide a first calcined stucco using a first set of calcining parameters; determining a phase content of the first calcined stucco according to a process as described herein; and based on the determined phase content, selecting a second calcium sulfate material different from the first calcium sulfate material and calcining it using a second set of calcining parameters to provide the stucco feedstock. In some embodiments, the second set of calcining parameters can be the same as the first set. In other embodiments, the second set of calcining parameters can be different from the first set. Any calcination parameters can be different, including those noted above. For example, in various embodiments, the second set has a different calcination temperature program than the first set. In various embodiments, the second set has a different calciner throughput or calcination time than the first set.

The second calcium sulfate material can differ from the first calcium sulfate material in a variety of manners. For example, in various embodiments, the second calcium sulfate material can have a different composition than the first calcium sulfate material, e.g., a different distribution of the various constituents (for example, substantially differing in relative amounts of one or more of calcium sulfate dihydrate (DH), calcium sulfate hemihydrate (HH), soluble calcium sulfate anhydrite (AIII); inert calcium sulfate (IN) and free moisture (FM), e.g., by more than 2 wt % or more than 5 wt % of one or more of these). In various embodiments, the second calcium sulfate material can be processed differently from the first calcium sulfate material. In various embodiments, the particle size distribution of the second calcium sulfate material can be different than that of the first calcium sulfate material. For example, in some embodiments, the second calcium sulfate material has a substantially different particle size distribution than the first material (e.g., a substantially different d50 value, for example, differing by at least 10% of the d50 of the first calcium sulfate material). In some embodiments, the second calcium sulfate material has a substantially larger d50 value than the d50 value of the first calcium sulfate material (e.g., by at least 10% of the d50 of the first calcium sulfate material). In other embodiments, the second calcium sulfate material has a substantially smaller d50 value than the d50 value of the first calcium sulfate material (e.g., by at least 10% of the d50 of the first calcium sulfate material). A smaller overall particle size (e.g., smaller d50) can result in a higher water demand and more energy expended in drying; a larger overall particle size (e.g., larger d50) would have the opposite effect. In some such embodiments, the second calcium sulfate material can have a substantially similar composition to the first calcium sulfate material (for example, not substantially differing in relative amounts of one or more of calcium sulfate dihydrate (DH), calcium sulfate hemihydrate (HH), soluble calcium sulfate anhydrite (AIII); inert calcium sulfate (IN) and free moisture (FM), e.g., by no more than 2 wt % or no more than 1 wt % of each of these).

The person of ordinary skill in the art will use material selection, material processing and calcination parameters to provide a desired stucco feedstock for a production process for a gypsum product. Generally, a high amount of hemihydrate and low amounts of dihydrate, anhydrite and inert materials are desirable, but the person of ordinary skill in the art will appreciate that processes can be tolerant to some degree of dihydrate, anhydrite and inert materials.

The stucco feedstocks provided by these processes can be used in the provision of gypsum products, using methods otherwise familiar to the person of ordinary skill in the art. For example, another aspect of the disclosure is production process for a gypsum product, the production process including: providing a stucco feedstock by the processes described above; hydrating the first stucco feedstock in an aqueous slurry; and allowing the slurry to set to form the gypsum product.

Knowledge of the phase content of a stucco feedstock used in the provision of a gypsum product can also be useful in determining parameters of the production process. For example, another aspect of the disclosure provides a production process for providing a gypsum product. The production process includes providing a stucco feedstock; determining a phase content of the stucco feedstock according to a process as described herein; hydrating the first stucco feedstock in an aqueous slurry; and allowing the slurry to set to form the gypsum product. Notably, the process also includes, based on the determined phase content of the first stucco feedstock, adjusting one or more parameters of the production process.

A production process typically includes forming an aqueous slurry from the stucco feedstock, then allowing that slurry to set to form a wet gypsum product, then drying the wet gypsum product to provide a dry gypsum product. Knowledge of the phase content of the stucco feedstock can provide the person of ordinary skill in the art with information helpful in adjusting a variety of parameters associated with such a process.

For example, knowledge of the phase content can be used by the person of ordinary skill in the art to adjust the content of the slurry. For example, in various embodiments, adjusting the content of the slurry includes selecting an amount of an accelerator (e.g., powdered calcium sulfate dihydrate or potassium sulfate) based on the determined phase content. More accelerator can be included in the slurry to cause a faster set of the slurry (e.g., when the amount of hemihydrate is relatively lower), or less accelerator can be included in the slurry to cause a slower set of the slurry (e.g., when the amount of hemihydrate is relatively higher). Similarly, in various embodiments, adjusting the content of the slurry includes selecting an amount of a set retarder (e.g., diethylenetriaminepentaacetic acid and salts thereof) based on the determined phase content. More set retarder can be included in the slurry to cause a slower set of the slurry (e.g., when relatively more hemihydrate is present), or less set retarder can be included in the slurry to cause faster set of the slurry (e.g., when relatively less hemihydrate is present). In various embodiments, adjusting the content of the slurry includes selecting the water demand of the slurry (i.e., the relative amount of water as compared to stucco feedstock) based on the determined phase content. Water demand can be, e.g., increased, e.g., when relatively more hemihydrate is present, but requires more energy in drying, or decreased, which requires less energy in drying. The overall density (e.g., measured as board weight for gypsum boards) can be adjusted, e.g., by tuning the amount of foaming agents (e.g., lauryl alcohol ether sulfates) or the foaming conditions (e.g., time/intensity of mixing air into the slurry) can be selected based on the determined phase content. Foaming can be increased to provide a higher density/board weight, which can increase strength but expend more energy in drying per unit volume of product, or a lower density/board weight, which can decrease strength but expend less energy in drying. The person of ordinary skill in the art, based on the disclosure herein, can provide a desired balance of energy expenditure (and thus cost expenditure in drying), board strength, and other properties by adjusting one or more parameters associated with the content of the slurry.

Moreover, parameters associated with drying can be adjusted based on knowledge of the phase content of the stucco feedstock. For example, parameters can be adjusted to provide relatively more drying or relatively less drying, e.g., dryer temperature (higher temperature to provide more drying; lower temperature to provide less drying); dryer humidity (lower humidity to provide more drying; higher humidity to provide less drying); and dryer throughput (less throughput/more residence time to provide more drying; more throughput/less residence time to provide less drying). Less drying can, for example, be provided when relatively more hemihydrate is present, as relatively more of the water originally present will be crystallized into dihydrate. The person of ordinary skill in the art, based on the disclosure herein, can provide a desired degree of drying to provide products that are sufficiently dried to provide desired mechanical strength, without over-drying to provide a process that operates at a desired degree of energy efficiency.

### Example 1

The Example that follows is illustrative of specific embodiments of the process of the disclosure, and various uses thereof. It is set forth for explanatory purposes only, and are not to be taken as limiting the scope of the disclosure.

### Data Acquisition

A large number of synthetic stucco samples were first prepared in order to build up a customized calcium sulfate database. Samples were prepared by mixing real industrial stuccos with pure calcium sulfate dihydrate, calcium sulfate hemihydrate, soluble calcium sulfate anhydrate, and inert calcium sulfate samples, and evaluated immediately by two standardized measurement routines: a dehydration test and a hydration test. These can be as described in Example 2, below. Raw experimental data were then labeled and saved into a local database. Since all experimental data in this database were collected under the same protocol, the changes in experimental results are directly related to the variations of the phase content in corresponding synthetic stucco samples.

### Preparation of Synthetic Stucco Samples

490 synthetic stucco samples were prepared by mixing industrial stucco samples with various amounts of calcium sulfate dihydrate, calcium sulfate hemihydrate, soluble calcium sulfate anhydrate, and/or inert calcium sulfate. Since the compositions of the industrial stucco samples were readily known, the phase content of the generated synthetic stucco samples could be easily calculated. Moreover, a conventional complete phase analysis (CPA) method (See Example 2, below) was used to confirm the accurate phase content of the first 183 groups of synthetic stucco samples. This quantitative analysis method has been proved with a high measurement accuracy, which provides more convinced and detailed information regarding the phase content of the synthetic stucco samples. Finally, the calculated phase contents were labeled as the “actual values” for each synthetic stucco sample, then stored into the local database. These “actual values” will be used as the benchmark to train the machine learning model in the following section.

Synthetic stucco samples were designed to represent actual industrial stuccos. Accordingly, they generally included 3.0% to 20.0% calcium sulfate dihydrate, 50.0% to 90.0% calcium sulfate hemihydrate, 0.0% to 30.0% soluble calcium sulfate anhydrate, and 5.0% to 10.0% inert calcium sulfate.

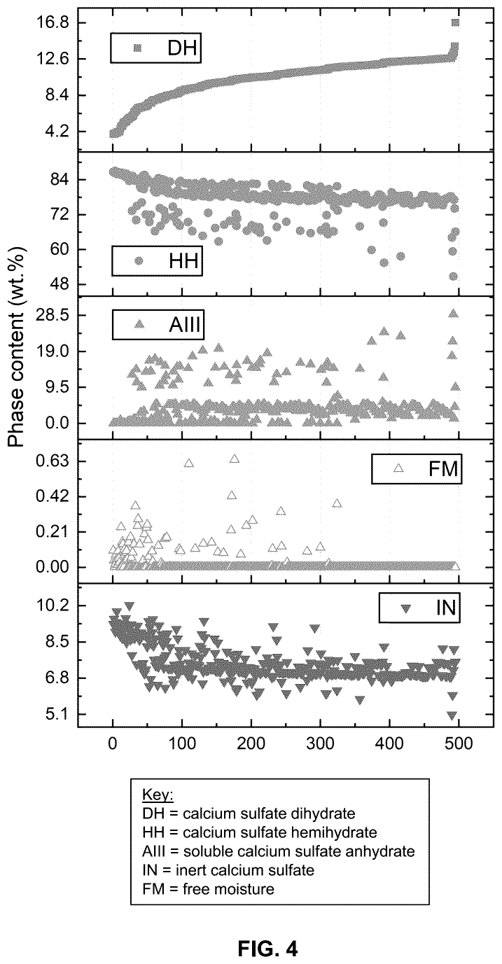

The actual phase contents of all 490 groups of synthetic stucco samples are given in FIG. 4. As shown, all phase contents were distributed within the desired ranges described above. This means that the as-prepared synthetic stucco samples are representative of industrial stucco products. It is also worth mentioning that free moisture content existed in some samples, but only at very low levels. It is known that free moisture cannot coexist with soluble calcium sulfate anhydrate because they quickly react with each other and form calcium sulfate hemihydrate. However, it is possible that a trace amount of free moisture was maintained in the calcium sulfate samples where soluble calcium sulfate anhydrate content is close to zero. Although the presence of free moisture is not common in industrial calcium sulfates, this situation is also marked and counted in the following machine learning model in an effort to improve the feasibility of the machine learning model in all possibilities.

### Dehydration Tests

Dehydration tests were conducted by monitoring the weight loss of synthetic stucco samples during dehydration using thermogravimetry analysis (TGA) methods. Dehydration tests were conducted using a COMPUTRAC® MAX® 5000XL moisture analyzer, and the dehydration temperature was set at 300° C. The weight loss (labeled as the moisture change) was recorded every eight seconds until reaching a plateau value. This plateau value was marked as the maximum moisture change value, which will be used in the following feature engineering step. Typically, the dehydration process took around 10 minutes to reach its plateau under this experimental setup.

### Hydration Tests

Hydration tests were conducted by measuring the temperature change after mixing the synthetic stucco sample with a certain amount of water. In this Example, hydration tests were conducted by adding 5 g of synthetic stucco sample into 5 mL of distilled water in a plastic tube. The temperature change was monitored by a thermocouple, while the whole system was kept in an isothermal box to minimize environmental disruption. The entire hydration process was recorded every second and typically took around 30 minutes to complete.

### Feature Engineering

The dehydration and hydration curves collected in this Example contained a large number of data points (e.g., ˜125 data points for each dehydration curve and ˜2000 data points for each hydration curve). In this project, each dehydration curve was parameterized using the value of the maximum moisture change value, as well as by fitting the dehydration rate (i.e., the rate of change of the evolution of moisture, as the first derivative of the raw dehydration curve) to a Gaussian function. Each hydration curve was fitted to a modified Gaussian function.

### Maximum Moisture Changes

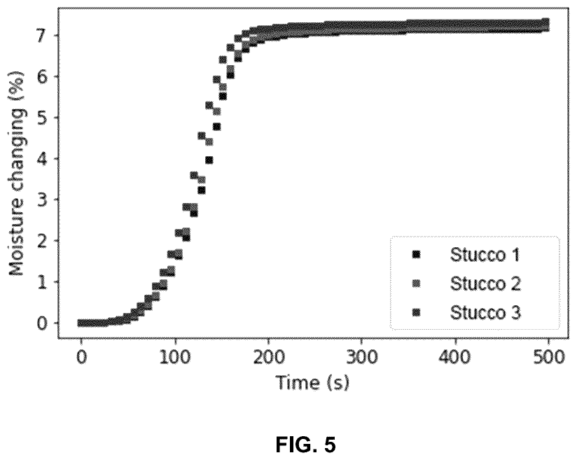

Examples for the dehydration curves are given in FIG. 5. For dehydration tests, one very important feature is the maximum moisture change value, which corresponds to the sum of the calcium sulfate dihydrate and calcium sulfate hemihydrate content in the synthetic stucco samples, as demonstrated by Error! Reference source not found. and Error! Reference source not found. Therefore, this value was determined as the first featurized parameter. Features could be extracted by finding the max values in each dehydration curve (labeled as “Cal_max”). All Cal_max values were then stored into a designed matrix of m×1 dimension (labeled as “featurized_parameter_1”), where m is the number of experimental groups.

### Fitting the Dehydration Curves

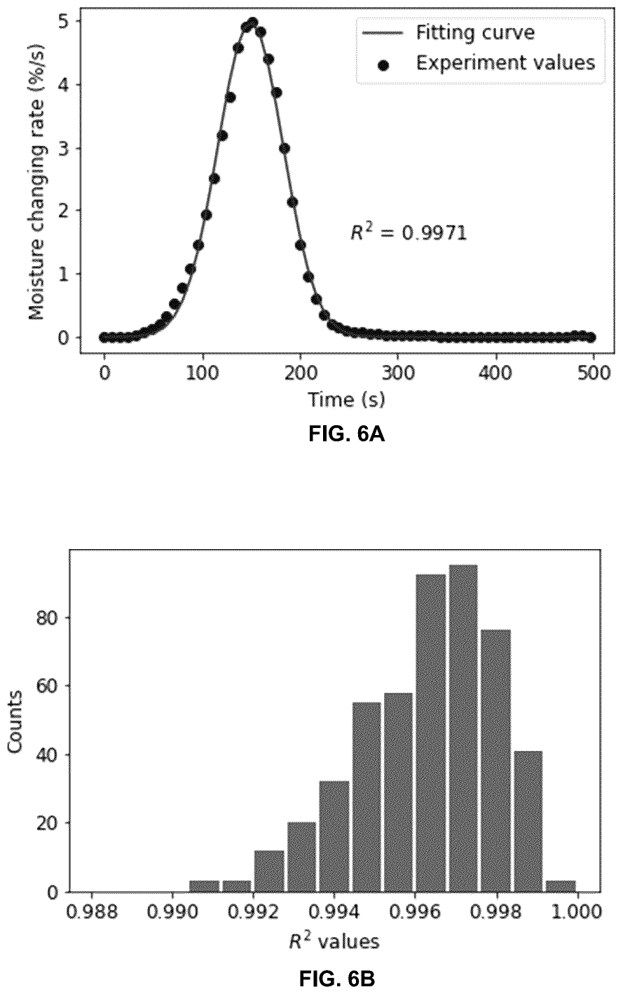

In an effort to represent the full dataset of the dehydration curve but also maintain a minimum number of featurized parameters, it was found that the dehydration rate (%/s) during dehydration tests (i.e., the first derivative of the dehydration weight loss curve) can be well-fitted with a Gaussian function as a function of time, t₁:

$$dehydration rate ( \% / s ) = a \cdot exp [ - {( \frac{{t}_{1} - b}{c} )}^{2} ] \qquad Eq . ( 1 )$$

where t₁ is the elapsed time of the dehydration test, and a, b, c are three non-zero independent moisture changing rate parameters of the Gaussian function. FIG. 6A gives an example of the experimental data (scatter points) and its corresponding fitting curve (line). It is demonstrated that the experimental values could be fitted precisely by a Gaussian function with an R² value of 0.9971. As shown in FIG. 6B, the R² value for every fitting curve is greater than 0.990, which means that all experimental dehydration data could be well represented. The resulting three parameters (cal_rate_a, cal_rate_b, and cal_rate_c) were also stored into a matrix named “featurized_parameter_2”, which possesses a dimension of m×3.

### Fitting the Hydration Curves

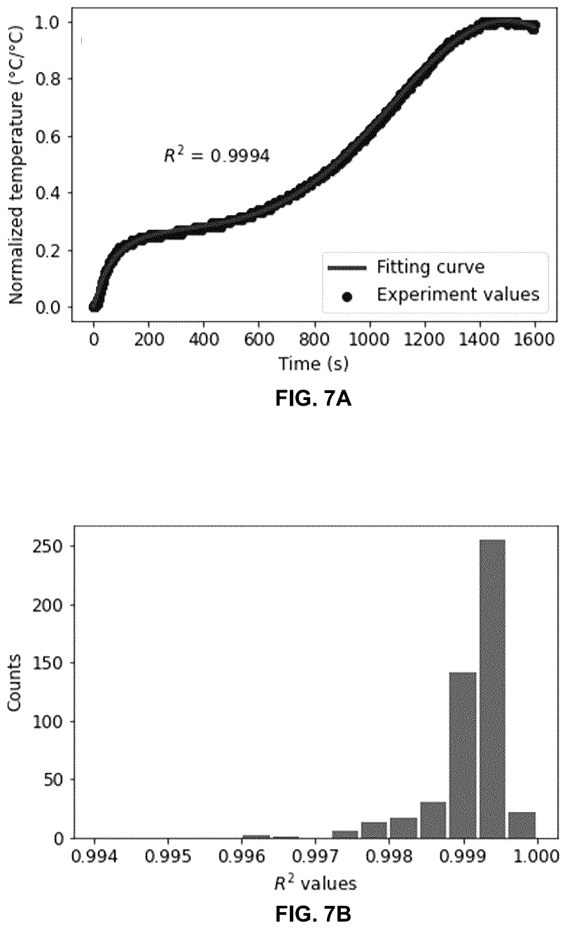

Similar to the case of dehydration curves, experimental data of hydration curves were engineered into six parameters using a modified Gaussian function as function of time t₂. In order to eliminate the impact of environmental temperature differences, the temperature was first normalized in each experiment group. In general, a typical hydration curve contains two major peaks, as shown in FIG. 7A. Therefore, the combination of positive-skewed and non-skewed Gaussian functions was considered to be the best fit for hydration test data by the following equation:

$$Normalized temperature ( ° C . \text{/°} C . ) = a \cdot exp [ - {( \frac{ln ( {t}_{2} ) - b}{c} )}^{2} ] + d \cdot exp [ - {( \frac{{t}_{2} - e}{f} )}^{2} ] \qquad Eq . ( 2 )$$

where t₂ is the elapsed time of calcination tests, and a, b, c, d, e, f are six independent temperature changing parameters. The first term in Eq. (2 is a positive-skewed Gaussian function, which was used to describe the first peak (at ˜100 s); on the other hand, the second term in Eq. (2 was designed to describe the second peak (at ˜1500 s) in the hydration curve. FIG. 7A gives an example of the experimental data during the hydration and its corresponding fitting curve. The R² value was as high as 0.9994, which means the fitting curve can represent the experiment data convincingly. FIG. 7B shows that the R² value for every fitting curve is greater than 0.995. The parameters (hyd_a, hyd_b, hyd_c, hyd_d, hyd_e, and hyd_f) were then stored into a matrix named “featurized_parameter_3” of m×6 dimension for the machine learning model.

### Machine Learning Model Using Artificial Neural Network (ANN)

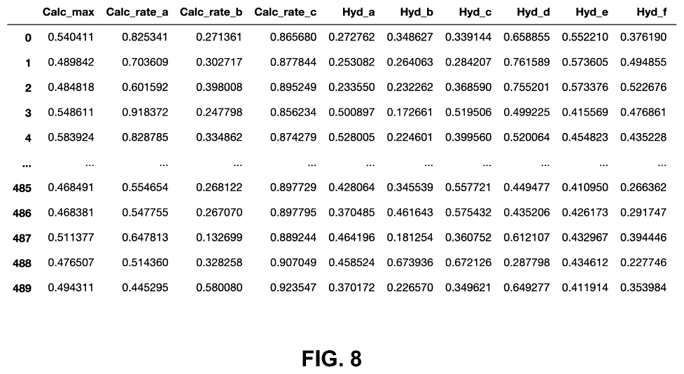

In the previous section, experimental data has been transformed into ten featurized parameters (i.e., a maximum moisture change, three independent moisture changing parameters, and six independent temperature changing parameters) and resulted in three matrices: featurized_parameter_1 (m×1), featurized_parameter_2 (m×3), and featurized_parameter_3 (m×6). The final input matrix (m×10) can be built up by combining these three matrices. An overview of the final featurized parameter matrix is provided in FIG. 8. In this final input matrix, parameters in each column were normalized within range [0, 1] in order to reduce computational cost and minimize biasing error.

An ANN algorithm was applied to the machine learning model in order to map the relationship between the featurized parameters and the phase content of synthetic stucco samples. The structure of the ANN is shown and described with respect to FIG. 3, above.

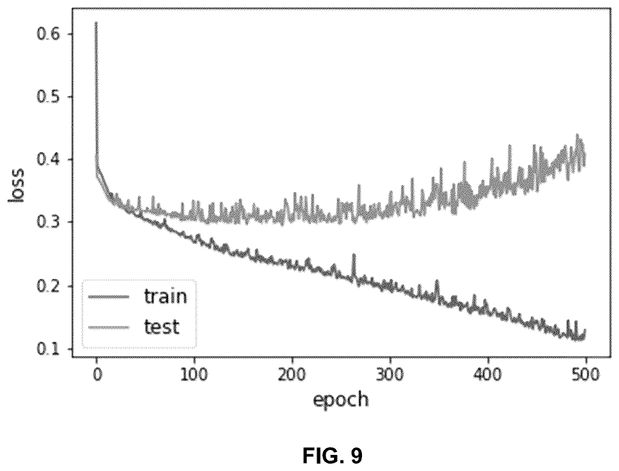

The ANN was trained by comparing the difference between the calculated values from the output layer and the actual values from sample preparation (i.e., values in FIG. 4). The differences between calculation and reality are described by the loss value (L) as described above. The machine learning model was trained for 500 epochs, and the changes of loss values are illustrated in FIG. 9. Experimental data were split into a training data set and a validation data set at an 80-to-20 ratio. The training data set was first fed into the machine learning model to train the weights in the ANN. As shown in FIG. 9 the loss values of the machine learning model kept decreasing over epochs for the training data set. This means that the model calculation became more accurate as the model iterated sufficient epochs. The validation data set was also fed into the trained machine learning model in each epoch with calculation of its corresponding loss values. The reason for using the validation data set is to examine whether the model is valid on an unseen data set. It was found that the loss value of the validation data set also decreased in the first 100 epochs, which means that the model became more accurate in this period on calculating the phase content of the validation data set. However, the loss value of the validation data set gradually increased after ˜300 epochs, even while loss value of the training data set kept decreasing, demonstrating the beginning of overfitting. Therefore, the early stopping technique was applied as the regularization to control the overfitting of the ANN. In this Example, it would be most appropriate to use as a final testing model the testing model from the epoch where the loss for the validation set data is substantially minimized, e.g., 300 epochs as in this example. Of course, other data sets would exhibit different overfitting behavior, and would exhibit different desirable stopping points to control overfitting.

### Performance Review

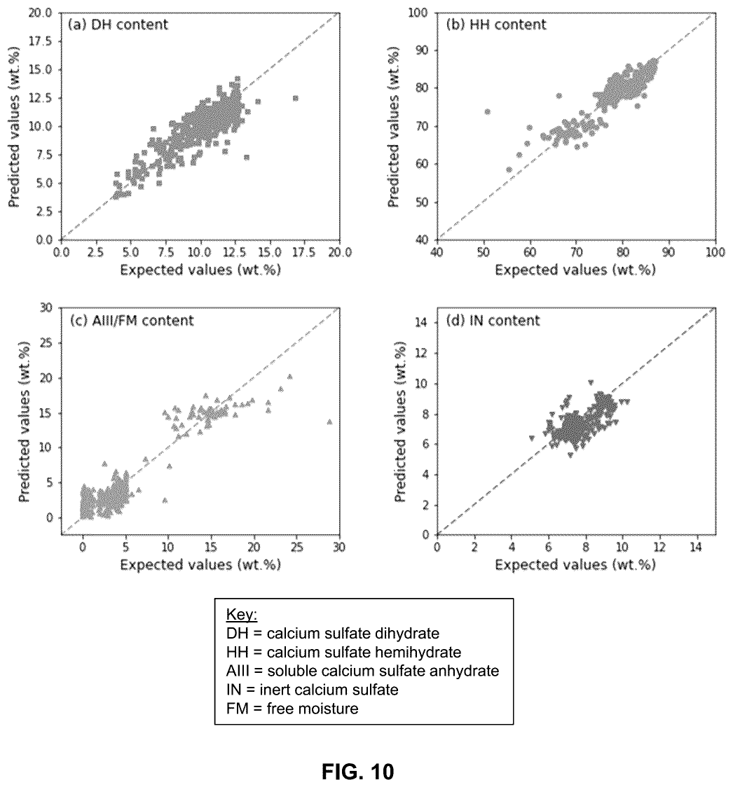

After finishing the training of the machine learning model, its performance was first visually evaluated by comparing the calculated values to the actual phase content values. FIG. 10 shows the comparison between the expected and calculated phase content values. Expected values (x-axis) of each phase content in the synthetic stucco samples were calculated from the sample preparation stage described in above, whereas the calculated values (y-axis) were obtained from the machine learning model. If the resulting data point sits on the diagonal line in each figure, it means that the calculated phase content is exactly the same as the expected value. The results in FIG. 10 demonstrated that most of the data points were very close to the diagonal line for each phase, except for very few outliers. These results provide a visual illustration that the current approach of machine learning model has an excellent accuracy on phase content analysis.

Finally, the performance of the machine learning model was evaluated quantitatively by two aspects: the root-mean-square-error (RMSE) of the calculated phase content value, and the accuracy of calculated free moisture content. Firstly, RMSE analysis indicates the differences between the calculated values from the machine learning model and the actual phase content values in synthetic stucco samples, as calculated by the following equation:

$$R M S E = \sqrt{\frac{1}{4} {\sum }_{i = 1}^{4} {( {phase}_{i}^{predicted} - {phase}_{i}^{expected} )}^{2}} \qquad Eq . ( 3 )$$

Based on this definition, a lower RMSE value would refer to a more accurate calculated of the machine learning model. In this study, the RMSE value of calculated stucco phase content in the test data set was calculated to be 2.2% when the model was trained for an iteration number of 300 epochs. Meanwhile, the accuracy of free moisture content calculation was determined to be 87.7% in this approach. Therefore, the low RMSE value and high type accuracy prove the concept that this machine learning model is an accurate and efficient approach to calculate the phase content in calcium sulfate materials.

Accordingly, the present inventors have developed a rapid and quantitative phase analysis method to determine the calcium sulfate phase content in calcium sulfate materials and products using a machine learning model combined with an artificial neural network (ANN) algorithm. This method has a simple and fast measurement routine, while only requiring conventional equipment for sample treatment. The calcium sulfate materials were subjected to a dehydration test, while its weight/moisture change was recorded during the dehydration test. The same calcium sulfate materials were also subjected to a hydration test by mixing the material with water for full hydration. Its temperature change was also recorded. After data acquisition and feature engineering, the featurized experimental data was fed into the trained machine learning model, where the calculation of the calcium sulfate materials corresponding phase content was easily calculated automatically. The accuracy of this analysis method was demonstrated by its low root-mean-square-error (RMSE) value on phase content calculation and high type accuracy on free moisture determination. Therefore, this method is considered to be suitable for the quality control of industrial calcium sulfate materials and products in large-scale operations.

### Example 2

Example 2 provides the so-called “CPA method” for determine the phase content of calcium sulfate samples. This Example describes a quantitative phase analysis that measures the amount of all calcium sulfate phases in a calcium sulfate sample. Broadly, the phase content is calculated using calcination weight loss and hydration weight gain of original, semi-hydrated, and fully hydrated samples. The uncomplicated sample preparation and conventional measurement routines make it suitable for on-site quality control measure in an industrial setting.

In gypsum wallboard manufacturing, calcium sulfate materials are generally provided in calcined form for use as a feedstock. These materials are typically fine white powder, and are often referred to as “stucco” after the hemihydrate phase that is typically desired. Depending on properties of the gypsum raw material and the calcination conditions, the stucco may contain different crystalline phases of the CaSO₄·xH₂O system including the main content of hemihydrate:
Calcium sulfate dihydrate (DH): It is possible to find some traces of dihydrate (“gypsum”) in the stucco due to incomplete calcination. Insufficient time or temperature in the calcinations kiln or large particle size of the gypsum raw materials are possible reasons for incomplete calcination. In the latter, the calcination to hemihydrate happens at the surface of the large particles while their cores can provide significant dihydrate content.
Calcium sulfate hemihydrate (HH): HH is the major constituent of the stucco mix and is formed from DH through an endothermic dehydration reaction typically between 110 and 120° C., as shown in the reaction (R-1):

$${CaSO}_{4} \cdot 2 {H}_{2} O {↔}^{Δ} {CaSO}_{4} \cdot \frac{1}{2} {H}_{2} O + \frac{3}{2} {H}_{2} O . \qquad ( R - 1 )$$

In general, HH can occur in two forms: α-form is predominant in wet methods (e.g., autoclaving) and β-form is predominant in dry methods (e.g., calcining). The β-form of HH is more relevant to building board production.
Soluble calcium sulfate anhydrite (AIII): Soluble anhydrite also has α and β forms that are prepared by further dehydration (typically 120-300° C.) of α and β hemihydrates, respectively.

$${CaSO}_{4} \cdot \frac{1}{2} {H}_{2} O {↔}^{Δ} {CaSO}_{4} + \frac{1}{2} {H}_{2} O . \qquad ( R - 2 )$$

Depending of the calcination conditions, AIII may directly form from DH without formations of the intermediate HH phase.

$${CaSO}_{4} {•2 H}_{2} O {↔}^{Δ} {CaSO}_{4} + 2 {H}_{2} O \qquad ( R - 3 )$$

AIII, also known as γ-CaSO₄, is very unstable and rehydrates to HH spontaneously if water vapor is present. Generally, the presence of AIII in the stucco is undesirable since it increases the water demand and makes it difficult to control the hydration process.
Insoluble calcium sulfate anhydrites (AII and AI): Further heating of AIII to temperatures higher than 300° C. results in the formation of an insoluble anhydrite known as β-CaSO₄ or AII. There is also a high temperature phase of anhydrite called α-CaSO₄ or AI, which is only stable above 1180° C. AII has a very limited hydration and AI shows no solubility in water. AII is relevant to the commercial applications of gypsum and is mainly categorized as the inert phase (IN) in this context.

In typical building board preparation, stucco is mixed with water and other solid or liquid additives to make a slurry, which is allowed to set (by hydration of hemihydrate to form interlocking dihydrate crystals) to form the “gypsum” board material. The phase content of the stucco greatly affects the properties of the slurry, such as water demand and setting time, that eventually govern the properties of the final drywall product. More specifically, controlling the setting time is important in modern drywall productions operating at high speed.

This Example provides a phase analysis method based on gravimetric and thermogravimetric analyses (GA and TGA) to quantify all phases in CaSO₄·xH₂O system. This method is a significant modification of Dweck and Lasota's method, described in J. Dweck, E. I. P. Lasota, Quality control of commercial plasters by thermogravimetry, Thermochim. Acta. 318 (1998) 137-142. doi:10.1016/S0040-6031(98)00338-4, which is hereby incorporated herein by reference in its entirety. Here, new samples, measurements, and equations to include AIII phase are included in the analysis. Sample preparation and measurements introduced in Example are simple enough to be performed in an industrial plant, i.e., without the need for a fully-equipped laboratory.

### Theory/Calculation

### Samples and Analyses

The quantitative phase analysis for a given stucco material is based on gravimetric and thermogravimetric phase analyses of three samples made from the stucco material as follows:
ORG (original): This is a sample taken from the stucco mix without any modification.
HUM (humidified and dried): This sample is made by conditioning the stucco in a humidity chamber. The humidity level, and duration of conditioning are tuned to make sure all AIII in the stucco converts to HH after the conditioning. After conditioning, the sample is dried at a temperature that is high enough to remove any free moisture (FM) but also is low enough to maintain the crystalline water.
HYD (hydrated and dried): This sample is made by full hydration of the stucco. Full hydration means there is enough water and time for all hydratable phases (HH and AIII) to convert to DH.

Two types of measurements are performed on the samples:
Gravimetric analysis (GA): This is basically measure of the weight change of HUM and HYD samples after conditioning. The output of this analysis is the weight gain of HUM and HYD samples reported in percentage as

$$\% Δ {W}^{g} = \frac{{W}_{f} - {W}_{0}}{{W}_{0}} \times 100 \qquad ( T - 1 )$$

where % ΔW^{g} is the percentage of weight gain during GA analysis, W_{f} and W₀ are the weight of samples with and without modification, respectively.
   Thermogravimetric analysis (TGA): This analysis measures the isothermal weight loss at a temperature high enough to remove all crystalline and free water. The absolute value of weight loss, % ΔW^{l}, is reported as percentages (similar to Equation (T-1)(T-1)) and measured for all three samples.

### Model Assumptions

The analysis method is based on two assumptions:
1. FM and AIII do not co-exist in the stucco. Since AIII can rapidly convert to HH in the presence of moisture, it is assumed that FM is stable only if there is no AIII in the stucco.
2. The only hydratable phases in the stucco are HH and AIII. In other words, the only partially hydrated calcium sulfate phase, CaSO₄·xH₂O, is HH (x=0.5). The stucco mix usually contains some paper debris that come from the recycles drywalls and can adsorb water. The amount of these impurities is very small and their effect on weight gain and loss can be neglected.

The consequence of the first assumption is that one needs to separate formulations for stucco containing AIII and FM. The presence of AIII or FM can be easily differentiated from the weight gain of the HUM sample (% ΔW_{HUM}^{g}). If there is AIII in the stucco, the weight of the sample should increase after conditioning in the humidity chamber. The crystalline water will not be removed in the following drying step however, the FM will be easily removed during the drying stage. So, one can write

$$\{ \begin{matrix}D + H + A + I = 100 if \% Δ {W}_{HUM}^{g} > 0 \\ D + H + F + I = 100 if \% Δ {W}_{HUM}^{g} < 0 \\ D + H + I = 100 if \% Δ {W}_{HUM}^{g} = 0\end{matrix} . \qquad ( T - 2 )$$

where D, H, A, F, and I are the weight percentages of DH, HH, AIII, FM, and IN phases in the stucco mix, respectively. This model has four unknowns (% ΔW_{HUM}^{g}=0 case is exceedingly rare and can be combined with other two cases), so four linear equations are required to solve for all unknowns. One equation is naturally given by Equation (T-2)(T-2) and other three equations can be provided by gravimetric and thermogravimetric analyses.

### Model Constants

This model uses absolute values of theoretical weight gain and losses of pure DH, HH, and AIII phases as constants. The notation used to represent these constants is C_{phase 2}^{phase 1} which is the absolute values of relative weight change when phase 1 transforms to phase 2. The numerical values of all constants used in the model can be easily calculated from the molecular weight of each phase of calcium sulfates. These values are also given in the Supplemental Information below.

### Thermogravimetric Phase Analysis (TGPA)

Three weight losses measured by thermogravimetric analysis for ORG, HUM, and HYD samples provide the remaining three equations needed to calculate phase contents. According to the model assumptions, AIII and FM cannot co-exist and each case should be treated separately.

Stucco Containing AIII (% ΔW_{HUM}^{g}>0)

The initial weight of all three samples can be calculated as

W₀=W_{DH}+W_{HH}+W_{AIII}+W_{IN}, (T-3)

where terms at the right side of the Equation (T-3) are the weights of each phase in the stucco. The weight loss measured by TGA for ORG sample is due to the dehydration of DH and HH phases (inverse reactions of (R-2) and (R-3)). So, one can simply write

% ΔW_{ORG}^{l}=C_{AIII}^{DH}×D+C_{AIII}^{HH}×H (T-4)

Note that phase percentages are calculated as

$$D = \frac{{W}_{DH}}{{W}_{0}} \times 1 \; 00 , \qquad ( T - 5 )$$

$$H = \frac{{W}_{HH}}{{W}_{0}} \times 100 ,$$

$$A = \frac{{W}_{AIII}}{{W}_{0}} \times 100 ,$$

$$F = \frac{{W}_{FM}}{{W}_{0}} \times 100 ,$$

$$I = \frac{{W}_{IN}}{{W}_{0}} \times 100 .$$

In Equation (T-5), numerators are the weight of each phase in the stucco and denominator is the initial weight of the sample (before conditioning and calcinations). When the stucco is conditioned in the humidifier to make HUM sample, all AIII content converts to HH, and the new weight of HH, W^{HH}′, can be written as

W_{HH}′=W_{HH}+W_{AIII}+C_{HH}^{AIII}×W_{AIII}. (T-6)

So, the weight of the HUM sample after conditioning reads as

W_{HUM}=W_{DH}+W_{HH}′+W_{IN}=W₀+C_{HH}^{AIII}×W_{AIII}. (T-7)

For the weight loss after calcination measured for HUM one can write

$$\% Δ {W}_{HUM}^{l} = \frac{Δ {W}_{HUM}}{{W}_{HUM}} \times 100 = \frac{{C}_{AIII}^{DH} \times {W}_{DH} + {C}_{AIII}^{HH} \times {W}_{HH}^{′}}{{W}_{0} + {C}_{H H}^{A l l l} \times {W}_{A l l l}} \times 100. \qquad ( T \text{-8)}$$

Dividing numerator and denominator of the fraction at the right side by W₀ and rearranging Equation (T-8) gives

$$\% Δ {W}_{HUM}^{l} = {C}_{AIII}^{DH} \times D + {C}_{AIII}^{HH} \times H + ( {C}_{AIII}^{HH} + {C}_{AIII}^{HH} \times {C}_{HH}^{AIII} - 0.01 \times \% Δ {W}_{HUM}^{l} \times {C}_{HH}^{AIII} ) \times A . \qquad ( T - 9 )$$

Similar calculations could be applied to HYD samples. In the HYD sample, all HH and AIII are transformed to DH, and the weight of the sample after conditioning can be written as

$${W}_{HYD} = {W}_{DH}^{′} + {W}_{IN} = {W}_{DH} + {W}_{HH} + {C}_{DH}^{HH} \times {W}_{HH} + {W}_{AIII} + {C}_{DH}^{AIII} \times {W}_{AIII} + {W}_{IN} = {W}_{0} + {C}_{DH}^{HH} \times {W}_{HH} + {C}_{DH}^{AIII} \times {W}_{AIII} . \qquad ( T - 103 )$$

For the weight loss measured for the HYD sample one can write

$$\% Δ {W}_{HYD}^{l} = \frac{Δ {W}_{HYD}}{{W}_{HYD}} \times 100 = \frac{{C}_{AIII}^{DH} \times {W}_{DH}^{′}}{{W}_{0} + {C}_{DH}^{HH} \times {W}_{HH} + {C}_{DH}^{AIII} \times {W}_{AIII}} \times 100. \qquad ( T - 11 )$$

As for the HUM sample, one can rewrite Equation (T(T-11)-11) as following linear relation.

$$\% Δ {W}_{HYD}^{l} = {C}_{AIII}^{DH} \times D + ( {C}_{AIII}^{DH} + {C}_{AIII}^{DH} \times {C}_{DH}^{HH} - 0.01 \times \% Δ {W}_{HYD}^{l} \times {C}_{DH}^{HH} ) \times H + ( {C}_{AIII}^{DH} + {C}_{AIII}^{DH} \times {C}_{DH}^{AIII} - 0.01 \times \% Δ {W}_{HYD}^{l} \times {C}_{DH}^{AIII} ) \times A \qquad ( T - 12 )$$

Equations (T-4), (T-8) and (T-12) build a system of three linear equations enabling one to solve for D, H, and A. Knowing three phase contents, I (inert phase content) can be easily calculated from Equation (T-2).

Stucco Containing Free Moisture (% ΔW_{HUM}^{g}<0)

When FM is presented in the stucco, the weight loss measured by TGA comes from dehydration of DH, HH, and the removal of FM. So, one can write

% ΔW_{ORG}^{l}=C_{AIII}^{DH}×D+C_{AIII}^{HH}×H+F. (T-13)

As explained above, conditioning stucco to make HUM sample removes the free moisture and the weight of the sample decreases. In the HYD sample, all HH converts to DH and FM is also removed during the drying step.

$${W}_{HUM} = {W}_{DH} + {W}_{HH} + {W}_{IN} = {W}_{0} - {W}_{FM} \qquad ( T - 14 )$$

$${W}_{HYD} = {W}_{DH} + {W}_{HH} + {C}_{DH}^{HH} \times {W}_{HH} + {W}_{IN} = {W}_{0} + {C}_{DH}^{HH} \times {W}_{HH} - {W}_{FM}$$

As for the stucco sample with AIII, one can write the following equations for the weight loss measured for HUM and HYD samples when the stucco contains FM.

$$\% Δ {W}_{HUM}^{l} = {C}_{AIII}^{DH} \times D + {C}_{AIII}^{HH} \times H + 0.01 \times \% Δ {W}_{HUM}^{l} \times F . \qquad ( T - 15 )$$

$$\% Δ {W}_{HYD}^{l} = {C}_{AIII}^{DH} \times D + ( {C}_{AIII}^{DH} + {C}_{AIII}^{DH} \times {C}_{DH}^{HH} - 0.01 \times \% Δ {W}_{HYD}^{l} \times {C}_{DH}^{HH} ) \times H + 0.01 \times \% Δ {W}_{HYD}^{l} \times F \qquad ( T - 16 )$$

Again, one can use Equations (T-13), (T-15) and (T-16) to build a system of linear equations to solve for D, H, and F. Both systems of linear equations developed for thermogravimetric phase analysis can be solved analytically. However, it is preferred to solve them as constraint optimization problems, in which phase contents are constrained between 0 and 100, to avoid nonphysical negative values for phase contents. The matrix representation of these systems of linear equations are shown in the Supplemental Information.

### Gravimetric Phase Analysis (GPA)

Since the weight gain can only be measured for HUM and HYD samples, gravimetric phase analysis lacks one equation to solve for all four phase contents. One can get the last equation from either of the three weight losses measured by TGA.

Stucco Containing AIII (% ΔW_{HUM}^{g}>0)

When the stucco contains AIII, weight of the sample increases after conditioning in the humidifier and the weight gain measured for the HUM sample is due to the conversion of AIII to HH. So, one can write

% ΔW_{HUM}^{g}=C_{HH}^{AIII}×A (T-17)

This equation simply gives the weight percent of the AIII phase. For the weight gain of the HYD sample one can write

% ΔW_{HYD}^{g}=C_{DH}^{HH}×H+C_{DH}^{AIII}×A (T-18)

Knowing A from Equation (T-17), Equation (T-18) readily gives H. One can then plug A and H into either of Equations (T-4), (T-9) and (T-12) to calculate D. As above, the weight percent of the inert phase is then calculated from Equation (T-2).
Stucco Containing Free Moisture (% ΔW_{HUM}^{g}<0)

If stucco contains free moisture, the free moisture is removed, and weight decreases after making HUM sample. So, the weight gain measured for HUM sample equals to the negative of the free moisture weight percent.

% ΔW_{HUM}^{g}=−F (T-19)

The free moisture also removed in the HYD sample and the weight gain can be written as

% ΔW_{HYD}^{g}=C_{DH}^{HH}×H−F (T-20)

One can again plug the F and H into either of Equations (T-13), (T-15) and (T-16) to calculate D, and then calculate I from Equation (T-2)(T-2).

Once ORG, HUM, and HYD samples are prepared for the thermogravimetric phase analysis, % ΔW_{HYD}^{g} and % ΔW_{HUM}^{g} can be easily measured by simple weight measurements. This means gravimetric analysis can be readily used to cross-check the accuracy of the thermogravimetric phase analysis and enhance the reliability of the calculations. Finally, one can summarize the phase analysis by categorizing all equations into two groups:
Phase analysis for stucco containing AIII that uses Equations (T-2), (T-4), (T-9), (T-12), (T-17) and (T-18) to calculate weight percent of DH, HH, AIII, and inert phases in the stucco.
Phase analysis for stucco containing free moisture that uses Equations (T-2), (T-13) (T-15), (T-16), (T-19) and (T-20) to calculate weight percent of DH, HH, free moisture, and inert phases in the stucco.

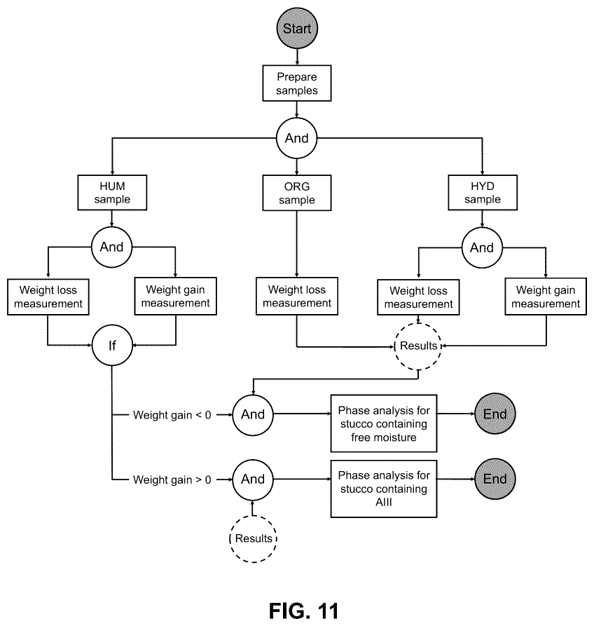

The flowchart shown in FIG. 11 summarizes the overall process for the phase analysis.

An example of a characterization method is provided below. The person of ordinary skill in the art can adapt this method as necessary.

### Materials and Methods

### Materials

Calcium sulfate dihydrate (ACS reagent 98%) and calcium sulfate hemihydrate (purum≥97%) were purchased from Sigma-Aldrich. The most reliable source for laboratory grade AIII is Drierite, which is an industrial desiccant made from calcinated gypsum. Drierite (without indicator, 8 mesh), was also purchased from Sigma-Aldrich. High purity AIII and HH samples were prepared from the DH reagent purchased from Sigma-Aldrich. To do this, DH was first calcined at 200° C. to obtain high purity AIII samples, which were conditioned in a humidifier (75% humidity, 45° C.) to make high purity HH. Both types of reagents (purchased and synthesized) were used to make synthetic “stucco” mixtures with variety of compositions. Insoluble calcium sulfate was used as the inert phase if needed. Also analyzed were industrial grade stucco samples provided by CertainTeed Gypsum, Inc. The industrial stucco sample is the mixture of different type of synthetic (FGDG) and recycled (WG) gypsums and used as a raw material in a building board production plant.

### Sample Preparation

To prepare the HUM sample, the stucco was first conditioned in a humidity chamber (LH-1.5, Associated Environmental Systems, MA, USA) with 75% humidity and 45° C. temperature overnight (at least 18 hours). The as-prepared HUM samples were then moved to an oven and dried at 45° C. for two hours to remove any possible free moisture. Finally, samples were cooled down to the room temperature (˜23° C.) at the vacuum oven. These HUM samples were sealed and stored in a desiccator for further use.

It is well known that AIII phase can easily rehydrate to HH even in a very low humidity. Although there are established industrial methods to condition stucco mix at high water pressure and temperature in order to reduce AIII content, conditions of full conversion of AIII to HH at the low pressures and temperatures have not been fully studied. The best humidity and temperature combination for full conversion of the AIII to HH in this study was achieved through trial-and-error—but the person of ordinary skill in the art can adapt the processes described here in their own analyses.

The HYD sample was made by full hydration of the stucco mix. To achieve full hydration, the stucco was mixed with enough water to completely submerge the powder and form the slurry. Samples were covered and let stand at the room temperature for 2 hours. Afterwards, samples were placed in the vacuum oven where they were dried at 45° C. overnight (at least 18 hours).

### Tests and Measurements

The HYD sample was made by full hydration of the stucco mix. To achieve full hydration, the stucco was mixed with enough water to completely submerge the powder and form the slurry. Samples were covered and let stand at the room temperature for 2 hours. Afterwards, samples were placed in the vacuum oven where they were dried at 45° C. overnight (at least 18 hours).

Weight gain of HUM and HYD samples after hydration were measured by a scale with reliability of 0.001 g (Denver Instrument PI-225D.3 Pinnacle Analytical Balance). Weight losses due to the full calcination (complete dehydration) was measured for ORG, HUM, and HYD samples using a TGA instrument, and/or a moisture analyzer. TA Instruments Q500 TGA instrument was used to perform 30 minute isothermal calcinations at 300° C. Samples tested by the moisture analyzer (Computrac® MAX® 5000XL by Arizona Instrument LLC) were heated to 300° C. and maintained at this temperature until the weight change rate become smaller than 0.1 g/s. All weight losses used in this particular study were measured with the moisture analyzer. While the TGA instrument can provides higher precision (0.1° C. and 0.1 μg) compared to the moisture analyzer (1° C. and 1 mg), it is believed the moisture analyzer is an appropriate tool for the stucco phase analysis for the following reasons:
1. The average difference between all phase contents calculated with two instruments were less than 1%.
2. The moisture analyzer is easy to set up and operate in industrial settings.
3. Samples analyzed by TGA are typically very small (˜50 mg) as compared to the amount that can be analyzed by the moisture analyzer (˜5 g). When testing an inhomogeneous mixture such as a sample of stucco, it is desirable to analyze a large enough amount to provide a meaningful statistical representation of the sample. In theory, repeating TGA measurements multiple times can address this issue. Bit considering the cost and time of TGA analysis in addition to the fact that the high precision provided by TGA is not necessary for this analysis, the moisture analyzer appears to be a highly suitable candidate for this application.
   For each of ORG, HUM, and HYD samples, at least three 10 g samples were prepared. The values reported for each phase content in this study are average values calculated from these three samples and error bars represent the standard deviation from the mean.

### Results and Discussion

### Single-Phase Samples

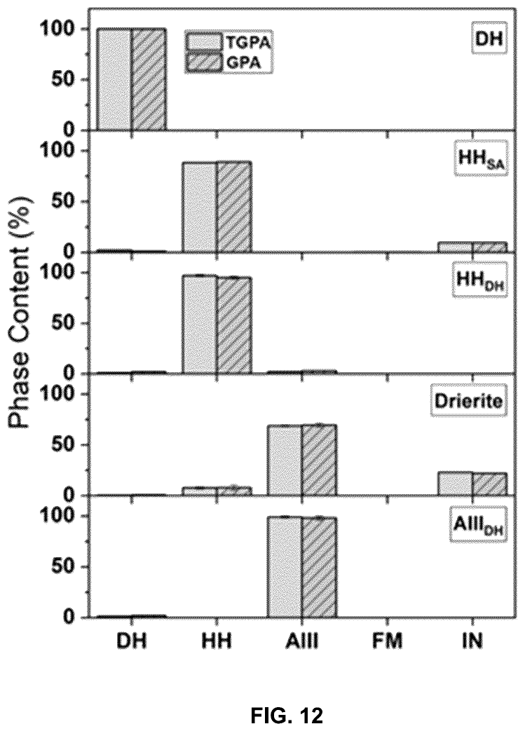

As explained in the previous section, two types of single-phase samples for HH and AIII were used. The first type were reagents purchased from Sigma-Aldrich directly, which are labeled as HH_{SA} and Drierite. The second-type samples are produced in-house from the purchased DH, using the procedure described above. These house-made HH and AIII samples were then labeled as HH_{DH} and AIII_{DH}, respectively. FIG. 12 shows the results of phase analysis performed on these samples; detailed information is also provided in Table S1 in the Supplemental Information.

FIG. 12 confirms that the DH reagent purchased from Sigma-Aldrich (DH) had a very high purity and contains almost 100% DH. The purchased HH_{SA} reagent, however, showed a HH content (˜89%) lower that the purity claimed for the sample (≥97%). The product sheet of these reagents shows that both Complexometric EDTA and X-RAY diffraction tests were used to characterize DH, but only the former was done to characterize HH_{SA}. Complexometric EDTA is a volumetric analysis that can measure calcium content, but it is not able to identify the phase that calcium is coming from. In other words, calcium in the HH_{SA} may come from hydratable or inert calcium-bearing phases other than hemihydrate. In the case of drierite, the purity or AIII content has not been reported, because drierite is often used as a desiccator and as a chemical reagent. The phase analysis described here detected about 69% AIII and about 22% IN content in the drierite. Some HH was also detected, which is most likely due to the moisture adsorbed from environment by some of the AIII. As shown in FIG. 12, one could synthesize high purity HH_{DH} and AIII_{DH} samples from the DH sample. By combining high purity samples, ten synthesized stucco samples were prepared; their phase analysis is explained in the next section.

### Synthesized Stucco Samples

The phase contents of the synthesized stucco samples were adjusted in a way to cover the range of the phase contents usually observed during the different stages of the industrial stucco preparation. Since single-phase samples with known phase contents were used, the phase contents of the synthesized stucco samples were also known. The expected phase contents of synthesized stucco samples are shown in Error! Reference source not found., below. In this table, the phase content columns refer to as the expected phase content in each synthesized stucco sample, whereas the corresponding formulation of each single-phase sample mix is also listed on the right-hand side. To be more specific, the values of phase content were calculated from the addition of each phase content in the single-phase sample mixture.

TABLE 1
Phase contents and the single-phase constituent
used to make synthesized stucco samples.

| Synthesized | Phase content (%) | | | | Single-phase samples in the mix (%) | | | | | |
|---|---|---|---|---|---|---|---|---|---|---|
| stucco | DH | HH | AIII | IN | DH | HH_{SA} | HH_{DH} | Drierite | AIII_{DH} | IN |
| 1 | 2.00 | 95.00 | 2.00 | 1.00 | 1.01 | 0 | 97.81 | 0 | 0.19 | 1.00 |
| 2 | 2.00 | 90.00 | 5.00 | 3.00 | 0.97 | 0 | 97.81 | 0 | 3.22 | 3.00 |
| 3 | 3.09 | 85.09 | 9.26 | 2.56 | 2.15 | 0 | 86.72 | 11.13 | 0 | 0 |
| 4 | 5.20 | 85.22 | 7.58 | 2.00 | 4.27 | 0 | 87.05 | 8.68 | 0 | 0 |
| 5 | 3.83 | 82.97 | 3.18 | 10.02 | 1.95 | 93.61 | 0 | 4.44 | 0 | 0 |
| 6 | 5.00 | 80.00 | 11.00 | 4.00 | 4.07 | 0 | 82.36 | 0 | 9.57 | 4.00 |
| 7 | 3.70 | 78.06 | 9.73 | 8.50 | 1.85 | 88.44 | 0 | 0 | 9.71 | 0 |
| 8 | 3.87 | 78.01 | 7.31 | 10.81 | 2.08 | 87.47 | 0 | 10.45 | 0 | 0 |
| 9 | 6.74 | 76.76 | 12.73 | 3.78 | 5.87 | 0 | 77.72 | 16.41 | 0 | 0 |
| 10 | 5.50 | 75.72 | 10.53 | 8.25 | 3.69 | 85.79 | 0 | 0 | 10.52 | 0 |

After preparing synthesized stucco samples, phase analysis was done, and the root mean squared error (RMSE) between expected and measured phase contents were calculated as

$$R M S E = \sqrt{\frac{1}{4} {{\sum }_{i = 1}}^{4} {( {phase}_{i}^{observed} - {phase}_{i}^{expected} )}^{2}} \qquad ( T - 21 )$$

The average RMSE for TGPA and GPA analysis for ten synthesized stucco samples were 0.45% and 1.06%, respectively. The lower accuracy of the GPA analysis is likely because this analysis uses two different types of measurements while all measurements used in TGPA are of the same type and precision. Overall, AIII phase showed the largest relative error between measured and expected values. This is most likely because of the nature of the AIII phase which is very unstable. AIII can rapidly adsorb moisture during the preparation of the synthesized stucco samples.

### Industrial Stucco Samples

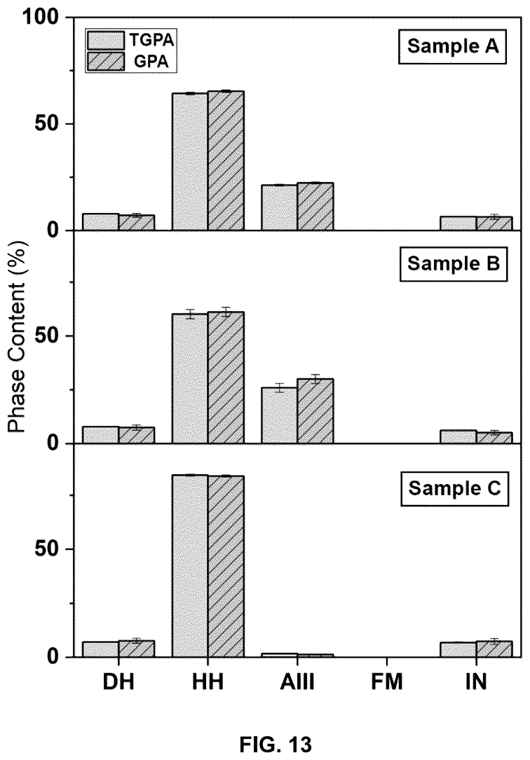

Industrial stucco samples were collected at different stages of the stucco preparation process at the midstream of a drywall production plant. Two samples (sample A & sample B) were collected at the initial stages of stucco preparation. In these two samples, calcination has been done to remove most of the DH content. Due to their specific preparation condition, sample A and sample B are expected to have a relatively high AIII content. The third sample (sample C) was collected from the output of the production plant, where AIII content had mostly been converted to HH. Phase analysis of these samples are shown in FIG. 13.

FIG. 13 shows that both calcination mills are operating in a similar way. There is a very small difference in the phase content of the stucco coming out of sample A and sample B. As expected, both sample A and sample B stuccos contain large amount of AIII because of over-calcination. Once these stuccos combined and conditioned in the mixer, the AIII content in the stucco decreases considerably. High amount of the AIII in stucco is tolerable as long as it helps reducing DH content. As shown in FIG. 13, DH content does not change after the calcination stage since high temperature is needed to remove DH.

### Mixture of Industrial Stucco Samples and Single-Phase Samples

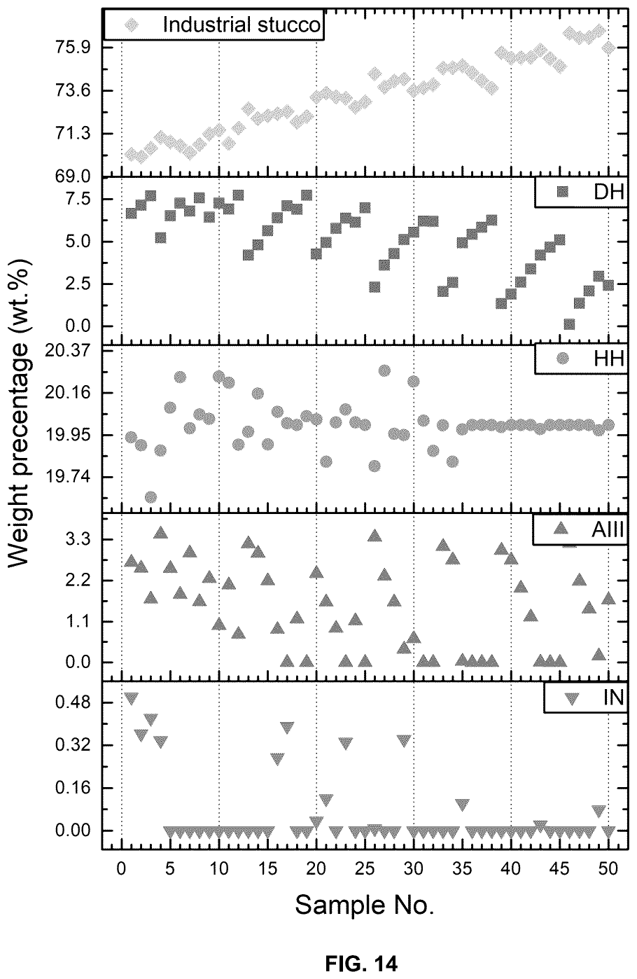

In order to further test the method for industrial stucco products, 50 groups of mixture samples were prepared by mixing industrial stucco samples with the single-phase samples. The primary motivation for this section of this study was to mimic the small differences of phase content in industrial stucco products by adding different amount of single-phase content manually, and then to confirm the sensitivity and reliability of the phase analysis procedure. The formation of the stucco-single phase mixture used in this study is shown in FIG. 14. More details of the mixtures are provided in Tables S2 & S3 in the Supplemental Information. As noted above, the average RMSE in TGPA analysis (0.45%) was smaller than that in GPA analysis (1.06%), which means that TGPA analysis is more accurate than GPA analysis at least within the range of stucco phase content in this study. Therefore, the following data are analyzed by the TGPA method.

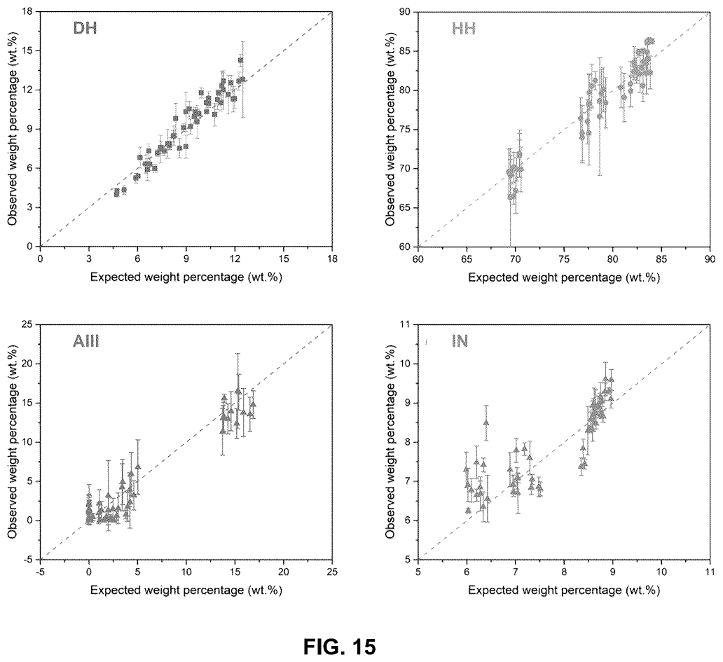

FIG. 15 shows the comparison between the expected values (x-axis) of each phase content in the stucco-single phase samples based on the sample preparation and the observed values (y-axis) obtained from the experiments and phase content analysis procedure. The diagonal in each figure refers to the situations, where the observed values match the expected values precisely. Deviations of the data point to the top-left corner mean that the experimental data provides a larger value of the corresponding phase than the expected values. In contrast, the deviation towards the bottom-right direction represents underestimated values through the analysis compared to expectations.

Results demonstrated that the phase analysis method developed in this study could provide reliable values to the phase content in the stucco-single phase mixture. The average RMSE value for these 50 groups of stucco-single phase samples is 1.14% as calculated by Equation (T-21), which is slightly higher than the average RMSE value obtained from the 10 groups of synthesized stucco samples (0.45%) described above. This is most likely due to the fact that industrial stucco samples are much more complicated than synthetic stucco samples and may have a higher variance during sampling. Nevertheless, the low value of RMSE still suggests a good quality of phase analysis using the procedure described here.

The calculated values of RMSE for DH, HH, AIII, and IN are 0.78%, 1.70%, 1.58%, and 0.60%, respectively. In general, the analysis of DH and IN contents were very reliable, whereas the RMSE values for HH and AIII contents were slightly higher but still convincing. It could be recognized from FIG. 15 that the data points of DH content are mostly lying around the diagonal line, indicating an excellent prediction of the anticipated values. The experimental values of IN content in FIG. 15 are slightly more scattered; however, this should be attributed to the relatively narrow scales of both x- and y-axes in this graph. It is worth noting that the observed IN content were commonly overestimated in the region where the expected IN content is low. Based on the discussion above, it is probable that the single-phase content (especially HH and AIII samples) used for the mixture may contain some IN content, which might lead to a low expected value of IN than reality. Therefore, the observed values of IN content are trustworthy in these scenarios. The main reason for DH and IN having good RMSEs in phase analysis tests is because of the fact that they are not sensitive to the free moisture in the air.

On the other hand, the slightly higher RMSEs of the HH and AIII contents compared to that of DH and IN contents are not unforeseen. As mentioned previously, AIII content is very sensitive to the free moisture and may transform into HH during sample preparation. Therefore, it is possible that some AIII was converted to HH during the experiments, which causes a decrease of AIII weight and the increase of HH weight simultaneously. This hypothesis is also supported by the result that the observed AIII content is usually smaller than that expected, when the expected value is readily small (FIG. 15). In fact, there were several groups with zero values of observed AIII content.

### CONCLUSIONS

An accurate phase analysis method that can measure the weight percent of all calcium sulfate phases in a stucco sample is provided. The phase content is calculated using weight loss and weight gain values respectively measured after calcination and hydration of three samples prepared from the stucco mix:
ORG sample: The original stucco without any conditioning.
HUM sample: The stucco mix conditioned at 45° C. and 75% humidity to convert all AIII to HH.
HYD sample: The stucco mix fully hydrated by adding water to convert all AIII and HH to DH.
   The method proposed in this study has significant advantages compared to the previously reported methods. Firstly, this method is capable to differentiate all soluble calcium sulfate phases and quantify their contents. Moreover, it requires simple sample preparation and measurements making is ideal for the on-site analysis at the industrial settings. The phase content of the stucco mix can greatly affect the production rate, water demand, and final product properties in the drywall production. The method proposed in this study can be used as a quality control measure at the midstream of the drywall production to control the quality of the stucco mix.

Of course, the person of ordinary skill in the art can adapt other methods to measure phase contents of known samples.

It will be apparent to those skilled in the art that various modifications and variations can be made to the processes and devices described here without departing from the scope of the disclosure. Thus, it is intended that the present disclosure cover such modifications and variations of this invention provided they come within the scope of the appended claims and their equivalents.

Additional aspects of the disclosure are provided by the following enumerated embodiments, which can be combined and permuted in any number and in any combination that is not technically or logically inconsistent.
Embodiment 1. A process for providing a computer-implemented system for determining a phase content of a test calcium sulfate material, the method comprising:
   providing a plurality of first reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
   for each of the plurality of first reference calcium sulfate samples, providing
      a first plurality of parameters associated with a dehydration weight loss curve of the first reference calcium sulfate sample, the first plurality of parameters comprising a first plurality of fit parameters associated with a fit of the dehydration weight loss curve of the first reference calcium sulfate sample, and
      a second plurality of parameters associated with a hydration temperature rise curve of the first reference calcium sulfate sample, the second plurality of parameters comprising a second plurality of fit parameters associated with a fit of the hydration curve of the first reference calcium sulfate sample,
   providing the first plurality of parameters and the second plurality of parameters for each of the first reference calcium sulfate samples to a computing device;
   via the computing device, correlating the first plurality of parameters and the second plurality of the parameters with the known contents of DH, HH, AIII, IN and FM of the first reference calcium sulfate samples, thereby providing a testing model that correlates the first plurality of parameters and the second plurality of parameters of a test calcium sulfate sample to contents of one or more of DH, HH, AIII, IN and FM of the test sample.
Embodiment 2. The process of embodiment 1, wherein the plurality of first reference calcium sulfate samples comprises at least 100 first reference calcium sulfate samples, e.g., at least 200 first reference calcium sulfate samples.
Embodiment 3. The process of embodiment 1, wherein the plurality of first reference calcium sulfate samples comprises at least 300 first reference calcium sulfate samples, e.g., at least 400 first reference calcium sulfate samples.
Embodiment 4. The process of any of embodiments 1-3, wherein each first reference calcium sulfate sample includes one or more of an industrial stucco product, phase-pure DH, phase-pure HH, phase-pure AIII and phase-pure IN.
Embodiment 5. The process of any of embodiments 1-4, wherein for at least 80% (e.g., at least 90% or at least 95%) of the plurality of first reference calcium sulfate samples, the known amount of DH is in the range of 3-20 wt %.
Embodiment 6. The process of any of embodiments 1-5, wherein for at least 80% (e.g., at least 90% or at least 95%) of the plurality of first reference calcium sulfate samples, the known amount of HH is in the range of 50-90 wt %.
Embodiment 7. The process of any of embodiments 1-6, wherein for at least 80% (e.g., at least 90% or at least 95%) of the plurality of first reference calcium sulfate samples, the known amount of AIII is up to 30 wt %.
Embodiment 8. The process of any of embodiments 1-7, wherein for at least 80% (e.g., at least 90% or at least 95%) of the plurality of first reference calcium sulfate samples, the known amount of IN is in the range of 5-10 wt %.
Embodiment 9. The process of any of embodiments 1-8, wherein the dehydration weight loss curve of each first reference calcium sulfate sample is provided by thermogravimetrically measuring weight loss of the first reference calcium sulfate sample at a temperature of at least 100° C., e.g., at least 200° C., or at least 250° C., e.g., in the range of 100-500° C., or 200-500° C., or 250-500° C., or 100-400° C., or 200-400° C., or 250-400° C.
Embodiment 10. The process of embodiment 9, wherein weight loss is measured over a time in the range of 5 to 30 minutes.
Embodiment 11. The process of embodiment 9 or embodiment 10, wherein the dehydration weight loss is desirably measured until the moisture change reaches a plateau.
Embodiment 12. The process of any of embodiments 1-11, wherein the fit of the dehydration weight loss curve is a fit of a moisture change rate as a function of time.
Embodiment 13. The process of embodiment 12, wherein the moisture change as a function of time is fit to a Gaussian function, e.g., the equation dehydration rate

$$( \% / s ) = a \cdot exp [ - {( \frac{{t}_{1} - b}{c} )}^{2} ] .$$

Embodiment 14. The process of any of embodiments 1-13, wherein first plurality of fit parameters comprises at least three fit parameters.
Embodiment 15. The process of any of embodiments 1-14, wherein the first plurality of parameters comprises a parameter associated with a maximum weight change of the dehydration weight loss curve.
Embodiment 16. The process of embodiment 15, wherein the parameter associated with a maximum weight change of the dehydration weight loss curve is a maximum weight change of the dehydration weight loss curve.
Embodiment 17. The process of any of embodiments 1-16, wherein the first plurality of parameters includes at least four parameters.
Embodiment 18. The process of any of embodiments 1-17, wherein the hydration temperature rise curve of each first reference calcium sulfate sample is provided by measuring the temperature rise as a function of time of a mixture of the first reference calcium sulfate sample and water.
Embodiment 19. The process of embodiment 18, wherein the temperature rise is measured over a period in the range of 15 minutes to 60 minutes, e.g., 15-45 minutes.
Embodiment 20. The process of embodiment 18 or embodiment 19, wherein a weight ratio of first reference calcium sulfate sample to water is in the range of 1:0.5-1:5, e.g., 1:0.5-1:2.
Embodiment 21. The process of any of embodiments 1-20, wherein the hydration temperature rise curve is fit to a combination of Gaussian functions, e.g., a combination of a positive-skewed Gaussian function and a non-skewed Gaussian function.
Embodiment 22. The process of embodiment 21, wherein the hydration temperature rise curve is fit to the equation temperature

$$change = u \cdot exp [ - {( \frac{ln ( {t}_{2} ) - v}{w} )}^{2} ] + x \cdot exp [ - {( \frac{{t}_{2} - y}{z} )}^{2} ] ,$$

e.g., where the temperature change is provided as a normalized temperature change by dividing temperature change by initial temperature.
Embodiment 23. The process of any of embodiments 1-22, wherein the second plurality of fit parameters comprises at least four parameters, e.g., at least five parameters.
Embodiment 24. The process of any of embodiments 1-22, wherein the second plurality of fit parameters comprises at least six parameters.
Embodiment 25. The process of any of embodiments 1-24, wherein the second plurality of parameters includes at least four parameters, e.g., at least five parameters.
Embodiment 26. The process of any of embodiments 1-24, wherein the second plurality of parameters includes at least six parameters.
Embodiment 27. The process according to any of embodiments 1-26, wherein the computing system includes a processor programmed to perform a machine learning algorithm.
Embodiment 28. The process according to embodiment 27, wherein the machine learning algorithm is an artificial neural network algorithm.
Embodiment 29. The process according to embodiment 28, wherein the artificial neural network has an input layer comprising at least one node for each parameter of the first plurality of parameters and the second plurality of parameters; at least two hidden layers having nodes associated with the input layer; and an output layer having at least five output nodes associated with the hidden layers.
Embodiment 30. The process according to embodiment 29, wherein the output nodes comprise regression nodes for each of DH, HH and IN.
Embodiment 31. The process according to embodiment 30, wherein the output nodes comprise two nodes that together relate to AIII and FM, e.g., a regression node for the total of AIII and FM, and a classification node classifying whether AIII or FM is present.
Embodiment 32. The process according to any of embodiments 28-31, wherein providing the testing model comprises training the artificial neural network algorithm through a plurality of epochs, e.g., at least 100 epochs, or at least 200 epochs, or at least 250 epochs.
Embodiment 33. The process according to embodiment 32, wherein in each epoch, training the artificial neural network algorithm comprises comparing differences between calculated values of the output layer and known values of one or more of DH, HH, IN, AIII and FM of the first reference calcium sulfate samples.
Embodiment 34. The process according to embodiment 33, wherein in each epoch, in response to differences between calculated values of the output layer and actual known values of one or more of DH, HH, IN, AIII and FM of the first reference calcium sulfate samples, weight associations are adjusted with respect to one or more of the parameters of the first plurality of parameters and the second plurality of parameters.
Embodiment 35. The process according to any of embodiments 1-34, further comprising
   providing a plurality of second reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
   for each of the plurality of second reference calcium sulfate samples, providing
      a third plurality of parameters associated with a dehydration weight loss curve of the second reference calcium sulfate sample, the third plurality of parameters comprising a third plurality of fit parameters associated with a fit of the dehydration weight loss curve of the second reference calcium sulfate sample, and
      a fourth plurality of parameters associated with a hydration temperature rise curve of the second reference calcium sulfate sample, the fourth plurality of parameters comprising a fourth plurality of fit parameters associated with a fit of the hydration curve of the second reference calcium sulfate sample;
   for each of the plurality of second reference calcium sulfate samples, providing the third plurality of parameters and the fourth plurality of parameters to the testing model; and
   using the testing model, determining the contents of one or more of DH, HH, AIII, IN and FM of each second reference calcium sulfate sample.
Embodiment 36. The method of embodiment 35, wherein providing the third plurality of parameters and the fourth plurality of parameters to the testing model and determining the contents of one or more of DH, HH, AIII, IN and FM of each second reference calcium sulfate sample is performed in a plurality of epochs.
Embodiment 37. The method of embodiment 36, further comprising providing a loss value (L) for each epoch, and selecting as a final testing model the testing model of an epoch where the loss value is substantially minimized.
Embodiment 38. A process for determining the phase content of a test calcium sulfate material, the method comprising:
   providing a dehydration weight loss curve of the test calcium sulfate material;
   providing a hydration temperature rise curve of the test calcium sulfate material;
   providing
      a first plurality of parameters associated with the dehydration weight loss curve of the test calcium sulfate sample, the first plurality of parameters comprising a first plurality of fit parameters associated with a fit of the dehydration weight loss curve of the test calcium sulfate sample, and
      a second plurality of parameters associated with the hydration temperature rise curve of the test calcium sulfate sample, the second plurality of parameters comprising a second plurality of fit parameters associated with a fit of the hydration curve of the test calcium sulfate sample;
   providing a testing model that correlates the first plurality of parameters and the second plurality of parameters of a test calcium sulfate sample to contents of one or more of DH, HH, AIII, IN and FM of the test sample;
   providing the first plurality of parameters and the second plurality of parameters to the testing model; and
   using the testing model, determining the contents of one or more of DH, HH, AIII, IN and FM of the test calcium sulfate sample.
Embodiment 39. The process according to embodiment 38, wherein the testing model is created by a method according to any of embodiments 1-36.
Embodiment 40. The process according to embodiment 38 or embodiment 39, wherein the amount of DH in the test calcium sulfate sample is in the range of 3-20 wt %.
Embodiment 41. The process of any of embodiments 38-40, wherein the amount of HH in the test calcium sulfate sample is in the range of 50-90 wt %.
Embodiment 42. The process of any of embodiments 38-41, wherein the amount of AIII in the test calcium sulfate sample is up to 30 wt %.
Embodiment 43. The process of any of embodiments 38-42, wherein the amount of IN in the test calcium sulfate sample is in the range of 5-10 wt %.
Embodiment 44. The process of any of embodiments 38-43, wherein the dehydration weight loss curve of the test sample is provided in substantially the same manner as the dehydration weight loss curves of the first reference calcium sulfate samples used in training the testing model.
Embodiment 45. The process of any of embodiments 38-44, wherein the dehydration weight loss curve of the test sample is provided by a method as described in any of embodiments 9-11.
Embodiment 46. The process of any of embodiments 38-45, wherein the first plurality of parameters of the test calcium sulfate sample are provided in substantially the same manner as the first plurality of parameters for the first reference calcium sulfate samples used in training the testing model.
Embodiment 47. The process of any of embodiments 38-46, wherein the first plurality of parameters are provided as described in any of embodiments 12-17.
Embodiment 48. The process of any of embodiments 38-47, wherein the hydration temperature rise curve of the test sample is provided in substantially the same manner as the hydration temperature rise curves of the first reference calcium sulfate samples used in training the testing model.
Embodiment 49. The process of any of embodiments 38-48, wherein the hydration temperature rise curve of the test sample is provided by a method as described in any of embodiments 18-20.
Embodiment 50. The process of any of embodiments 38-49, wherein the second plurality of parameters of the test calcium sulfate sample are provided in substantially the same manner as the second plurality of parameters for the second reference calcium sulfate samples used in training the testing model.
Embodiment 51. The process of any of embodiments 38-50, wherein the second plurality of parameters are provided as described in any of embodiments 21-26.
Embodiment 52. A process for providing a stucco feedstock, the process comprising
   providing a first stucco material;
   determining a phase content of the first stucco material according to the process of any of embodiments 38-51; and
   based on the determined phase content, modifying the first stucco material to provide a stucco feedstock with a desired phase content.
Embodiment 53. The process of embodiment 52, wherein the modification of the first stucco material comprises a combination of the first stucco material with one or more second stucco materials each having a different phase content from the first stucco material, to provide a stucco feedstock with a desired phase content.
Embodiment 54. The process of embodiment 52 or embodiment 53, wherein the modification of the first stucco material comprises calcining the first stucco material.
Embodiment 55. The process of embodiment any of embodiments 52-54, wherein the modification of the first stucco material comprises adding moisture to the first stucco material.
Embodiment 56. The process of any of embodiments 52-55, wherein the modification of the first stucco material comprises adjusting a particle size distribution (e.g., by adjusting grinding conditions) of the first stucco material.
Embodiment 57. A process for providing a stucco feedstock, the process comprising
   calcining a first sample of a calcium sulfate material to provide a first calcined stucco using a first set of calcining parameters;
   determining a phase content of the first calcined stucco according to the process of any of embodiments 38-51;
   based on the determined phase content, calcining a second sample of the calcium sulfate material using a second set of calcining parameters different from the first set of calcining parameters to provide the stucco feedstock.
Embodiment 58. The process of embodiment 57, wherein the second set has a higher calcination temperature program than the first set.
Embodiment 59. The process of embodiment 57, wherein the second set has a lower calcination temperature program than the first set.
Embodiment 60. The process of any of embodiments 57-59, wherein the second set has a higher calcination humidity than the first set.
Embodiment 61. The process of any of embodiments 57-59, wherein the second set has a lower calcination humidity than the first set.
Embodiment 62. The process of any of embodiments 57-61, wherein the second set has a higher calciner throughput than the first set.
Embodiment 63. The process of any of embodiments 57-61, wherein the second set has a lower calciner throughput than the first set.
Embodiment 64. The process of any of embodiments 57-63, wherein the second set has a longer calcination time than the first set.
Embodiment 65. The process of any of embodiments 57-63, wherein the second set has a shorter calcination time than the first set.
Embodiment 66. A process for providing a stucco feedstock, the process comprising
   calcining a first calcium sulfate material to provide a first calcined stucco using a first set of calcining parameters;
   determining a phase content of the first calcined stucco according to the process of any of embodiments 38-51;
   based on the determined phase content, selecting a second calcium sulfate material different from the first calcium sulfate material and calcining it using a second set of calcining parameters to provide the stucco feedstock.
Embodiment 67. The process according to embodiment 66, wherein the second calcium sulfate material has a different composition than the first calcium sulfate material.
Embodiment 68. The process according to embodiment 67, wherein the second calcium sulfate material has substantially different relative amounts of one or more of calcium sulfate dihydrate (DH), calcium sulfate hemihydrate (HH), soluble calcium sulfate anhydrite (AIII); inert calcium sulfate (IN) and free moisture (FM) than the first calcium sulfate material, e.g., differing by more than 2 wt % or more than 5 wt % of one or more of these.
Embodiment 69. The process according to any of embodiments 66-68, wherein the second calcium sulfate material is processed differently from the first calcium sulfate material.
Embodiment 70. The process according to any of embodiments 66-69, wherein the second calcium sulfate material has a substantially different particle size distribution than the first material (e.g., a substantially different d50 value, for example, differing by at least 10% of the d50 of the first calcium sulfate material).
Embodiment 71. The process according to embodiment 70, wherein the second calcium sulfate material has a substantially larger d50 value than the d50 value of the first calcium sulfate material (e.g., by at least 10% of the d50 of the first calcium sulfate material).
Embodiment 72. The process according to embodiment 70, wherein the second calcium sulfate material has a substantially smaller d50 value than the d50 value of the first calcium sulfate material (e.g., by at least 10% of the d50 of the first calcium sulfate material).
Embodiment 73. The process according to any of embodiments 66-72, wherein the second calcium sulfate material has a substantially similar composition to the first calcium sulfate material (for example, not substantially differing in relative amounts of one or more of calcium sulfate dihydrate (DH), calcium sulfate hemihydrate (HH), soluble calcium sulfate anhydrite (AIII); inert calcium sulfate (IN) and free moisture (FM), e.g., by no more than 2 wt % or no more than 1 wt % of each of these).
Embodiment 74. The process of any of embodiments 66-73, wherein the second set has a higher calcination temperature program than the first set.
Embodiment 75. The process of any of embodiments 66-73, wherein the second set has a lower calcination temperature program than the first set.
Embodiment 76. The process of any of embodiments 66-75, wherein the second set has a higher calcination humidity than the first set.
Embodiment 77. The process of any of embodiments 66-75, wherein the second set has a lower calcination humidity than the first set.
Embodiment 78. The process of any of embodiments 66-77, wherein the second set has a higher calciner throughput humidity than the first set.
Embodiment 79. The process of any of embodiments 66-77, wherein the second set has a lower calciner throughput than the first set.
Embodiment 80. The process of any of embodiments 66-79, wherein the second set has a longer calcination time than the first set.
Embodiment 81. The process of any of embodiments 66-79, wherein the second set has a shorter calcination time than the first set.
Embodiment 82. A production process for a gypsum product, the production process comprising:
   providing a stucco feedstock by the process of any of embodiments 52-81;
   hydrating the first stucco feedstock in an aqueous slurry; and
   allowing the slurry to set; and
   drying the set slurry to form the gypsum product.
Embodiment 83. A production process for providing a gypsum product, comprising:
   providing a first stucco feedstock;
   determining a phase content of the first stucco feedstock according to the process of any of embodiments 38-51;
   hydrating the first stucco feedstock in an aqueous slurry;
   allowing the slurry to set; and
   drying the set slurry to form the gypsum product,
   the process further comprising, based on the determined phase content of the first stucco feedstock, adjusting one or more parameters of the production process.
Embodiment 84. The production process of embodiment 83, wherein adjusting one or more parameters of the production process comprises adjusting the content of the slurry,
Embodiment 85. The production process of embodiment 84, wherein adjusting the content of the slurry includes selecting an amount of an accelerator (e.g., powdered calcium sulfate dihydrate or potassium sulfate) based on the determined phase content.
Embodiment 86. The production process of embodiment 84 or embodiment 85, wherein adjusting the content of the slurry includes selecting an amount of a set retarder (e.g., diethylenetriaminepentaacetic acid and salts thereof) based on the determined phase content.
Embodiment 87. The production process of any of embodiments 83-86, wherein adjusting the content of the slurry includes selecting the water demand of the slurry (i.e., the relative amount of water as compared to stucco feedstock) based on the determined phase content.
Embodiment 88. The production process of any of embodiments 83-87, wherein adjusting the content of the slurry includes selecting the density (e.g., as a board weight) of the gypsum product based on the determined phase content.
Embodiment 89. The production process of any of embodiments 82-88, wherein adjusting one or more parameters of the production process comprises adjusting the one or more parameters associated with drying.

We claim:

1. A process for providing a computer-implemented system for determining a phase content of a test calcium sulfate material, the method comprising:
providing a plurality of first reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
for each of the plurality of first reference calcium sulfate samples, providing
a first plurality of parameters associated with a dehydration weight loss curve of the first reference calcium sulfate sample, the first plurality of parameters comprising a first plurality of fit parameters associated with a fit of the dehydration weight loss curve of the first reference calcium sulfate sample, and
a second plurality of parameters associated with a hydration temperature rise curve of the first reference calcium sulfate sample, the second plurality of parameters comprising a second plurality of fit parameters associated with a fit of the hydration curve of the first reference calcium sulfate sample,
providing the first plurality of parameters and the second plurality of parameters for each of the first reference calcium sulfate samples to a computing device;
via the computing device, correlating the first plurality of parameters and the second plurality of the parameters with the known contents of DH, HH, AIII, IN and FM of the first reference calcium sulfate samples, thereby providing a testing model that correlates the first plurality of parameters and the second plurality of parameters of a test calcium sulfate sample to contents of one or more of DH, HH, AIII, IN and FM of the test sample.

2. The process of claim 1, wherein the plurality of first reference calcium sulfate samples comprises at least 300 first reference calcium sulfate samples.

3. The process of claim 1, wherein
for at least 80% of the plurality of first reference calcium sulfate samples, the known amount of DH is in the range of 3-20 wt %.
for at least 80% of the plurality of first reference calcium sulfate samples, the known amount of HH is in the range of 50-90 wt %.
for at least 80% of the plurality of first reference calcium sulfate samples, the known amount of AIII is up to 30 wt %.
for at least 80% of the plurality of first reference calcium sulfate samples, the known amount of IN is in the range of 5-10 wt %.

4. The process of claim 1, wherein the dehydration weight loss curve of each first reference calcium sulfate sample is provided by thermogravimetrically measuring weight loss of the first reference calcium sulfate sample at a temperature of at least 100° C.

5. The process of claim 1, wherein the fit of the dehydration weight loss curve is a fit of a moisture change rate as a function of time.

6. The process of claim 1, wherein the first plurality of parameters includes at least four parameters.

7. The process of claim 1, wherein the hydration temperature rise curve of each first reference calcium sulfate sample is provided by measuring the temperature rise as a function of time of a mixture of the first reference calcium sulfate sample and water.

8. The process of claim 1, wherein the hydration temperature rise curve is fit to a combination of a positive-skewed Gaussian function and a non-skewed Gaussian function.

9. The process of claim 1, wherein the second plurality of fit parameters comprises at least five parameters.

10. The process of claim 1, wherein the second plurality of parameters includes at least five parameters.

11. The process of claim 1, wherein the computing system includes a processor programmed to perform a machine learning algorithm that is an artificial neural network algorithm.

12. The process of claim 1, further comprising
providing a plurality of second reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
for each of the plurality of second reference calcium sulfate samples, providing
a third plurality of parameters associated with a dehydration weight loss curve of the second reference calcium sulfate sample, the third plurality of parameters comprising a third plurality of fit parameters associated with a fit of the dehydration weight loss curve of the second reference calcium sulfate sample, and
a fourth plurality of parameters associated with a hydration temperature rise curve of the second reference calcium sulfate sample, the fourth plurality of parameters comprising a fourth plurality of fit parameters associated with a fit of the hydration curve of the second reference calcium sulfate sample;
for each of the plurality of second reference calcium sulfate samples, providing the third plurality of parameters and the fourth plurality of parameters to the testing model; and
using the testing model, determining the contents of one or more of DH, HH, AIII, IN and FM of each second reference calcium sulfate sample.

13. A process for determining the phase content of a test calcium sulfate material, the method comprising:
providing a dehydration weight loss curve of the test calcium sulfate material;
providing a hydration temperature rise curve of the test calcium sulfate material;
providing
a first plurality of parameters associated with the dehydration weight loss curve of the test calcium sulfate sample, the first plurality of parameters comprising a first plurality of fit parameters associated with a fit of the dehydration weight loss curve of the test calcium sulfate sample, and
a second plurality of parameters associated with the hydration temperature rise curve of the test calcium sulfate sample, the second plurality of parameters comprising a second plurality of fit parameters associated with a fit of the hydration curve of the test calcium sulfate sample;
providing a testing model that correlates the first plurality of parameters and the second plurality of parameters of a test calcium sulfate sample to contents of one or more of DH, HH, AIII, IN and FM of the test sample;
providing the first plurality of parameters and the second plurality of parameters to the testing model; and
using the testing model, determining the contents of one or more of DH, HH, AIII, IN and FM of the test calcium sulfate sample.

14. The process according to claim 13, wherein the testing model is created by a method comprising:
providing a plurality of first reference calcium sulfate samples, each having a known content of calcium sulfate dihydrate (DH), calcium sulfate hemihydrate (HH), soluble calcium sulfate anhydrite (AIII); inert calcium sulfate (IN) and free moisture (FM);
for each of the plurality of first reference calcium sulfate samples, providing
a first plurality of parameters associated with a dehydration weight loss curve of the first reference calcium sulfate sample, the first plurality of parameters comprising a first plurality of fit parameters associated with a fit of the dehydration weight loss curve of the first reference calcium sulfate sample, and
a second plurality of parameters associated with a hydration temperature rise curve of the first reference calcium sulfate sample, the second plurality of parameters comprising a second plurality of fit parameters associated with a fit of the hydration curve of the first reference calcium sulfate sample,
providing the first plurality of parameters and the second plurality of parameters for each of the first reference calcium sulfate samples to a computing device; and
via the computing device, correlating the first plurality of parameters and the second plurality of the parameters with the known contents of DH, HH, AIII, IN and FM of the first reference calcium sulfate samples, thereby providing a testing model that correlates the first plurality of parameters and the second plurality of parameters of a test calcium sulfate sample to contents of one or more of DH, HH, AIII, IN and FM of the test sample

15. A process for providing a stucco feedstock, the process comprising providing a first stucco material;
determining a phase content of the first stucco material according to the process of claim 13; and
based on the determined phase content, modifying the first stucco material to provide a stucco feedstock with a desired phase content.

16. A process for providing a stucco feedstock, the process comprising
calcining a first sample of a calcium sulfate material to provide a first calcined stucco using a first set of calcining parameters;
determining a phase content of the first calcined stucco according to the process of claim 13; and
based on the determined phase content, calcining a second sample of the calcium sulfate material using a second set of calcining parameters different from the first set of calcining parameters to provide the stucco feedstock.

17. A process for providing a stucco feedstock, the process comprising
calcining a first calcium sulfate material to provide a first calcined stucco using a first set of calcining parameters;
determining a phase content of the first calcined stucco according to the process of claim 13;
based on the determined phase content, selecting a second calcium sulfate material different from the first calcium sulfate material and calcining it using a second set of calcining parameters to provide the stucco feedstock.

18. A production process for a gypsum product, the production process comprising:
providing a stucco feedstock by the process of claim 15;
hydrating the first stucco feedstock in an aqueous slurry; and
allowing the slurry to set; and
drying the set slurry to form the gypsum product.

19. A production process for a gypsum product, the production process comprising:
providing a stucco feedstock by the process of claim 16;
hydrating the first stucco feedstock in an aqueous slurry; and
allowing the slurry to set; and
drying the set slurry to form the gypsum product.

20. A production process for a gypsum product, the production process comprising:
providing a stucco feedstock by the process of claim 17;
hydrating the first stucco feedstock in an aqueous slurry; and
allowing the slurry to set; and
drying the set slurry to form the gypsum product.

21. A production process for providing a gypsum product, comprising:
providing a first stucco feedstock;
determining a phase content of the first stucco feedstock according to the process of claim 13;
hydrating the first stucco feedstock in an aqueous slurry;
allowing the slurry to set; and
drying the set slurry to form the gypsum product,
the process further comprising, based on the determined phase content of the first stucco feedstock, adjusting one or more parameters of the production process.

22. The production process of claim 21, wherein adjusting one or more parameters of the production process comprises adjusting the content of the slurry, wherein adjusting the content of the slurry includes one or more of
selecting an amount of an accelerator based on the determined phase content;
selecting an amount of a set retarder based on the determined phase content; and
selecting the water demand of the slurry based on the determined phase content.

23. The production process of claim 21, wherein adjusting one or more parameters of the production process comprises adjusting the one or more parameters associated with drying.
